SEQUENCE LISTING (9 sequences)

SEQ ID NO: 1 - protein, 476 aa, Escherichia coli
MITSALHRAADWAKSVFSSAALGDPRRTARLVNVAAQLAKYSGKSITISSEGSEAMQEGAYRFIRNPNVSAEAIRKAGAMQTVKLAQEFPELLAIEDTTSLSYRHQVAEELGKLGSIQDKSRGWWVHSVLLLEATTFRTVGLLHQEWWMRPDDPADADEKESGKWLAAAATSRLRMGSMMSNVIAVCDREADIHAYLQDKLAHNERFVVRSKHPRKDVESGLYLYDHLKNQPELGGYQISIPQKGVVDKRGKRKNRPARKASLSLRSGRITLKQGNITLNAVLAEEINPPKGETPLKWLLLTSEPVESLAQALRVIDIYTHRWRIEEFHKAWKTGAGAERQRMEEPDNLERMVSILSFVAVRLLQLRESFTLPQALRAQGLLKEAEHVESQSAETVLTPDECQLLGYLDKGKRKRKEKAGSLQWAYMAIARLGGFMDSKRTGIASWGALWEGWEALQSKLDGFLAAKDLMAQGIKI

SEQ ID NO: 2 - protein, 662 aa, Escherichia coli
MKEWYTAKELLGLAGLPKQATNITRKAQREGWEFRQVAGTKGVSFEFNIKSFPVALRAEILLQQGRIETSQGYFEIARPTLEAHDYDREALWSKWDNASDSQRRLAEKWLPAVQAADEMLNQGISTKTAFATVAGHYQVSASTLRDKYYQVQKFAKPDWAAALVDGRGASRRNVHKSEFDEDAWQFLIADYLRPEKPAFRKCYERLELAAREHGWSIPSRATAFRRIQQLDEAMVVACREGEHALMHLIPAQQRTVEHLDAMQWINGDGYLHNVFVRWFNGDVIRPKTWFWQDVKTRKILGWRCDVSENIDSIRLSFMDVVTRYGIPEDFHITIDNTRGAANKWLTGGAPNRYRFKVKEDDPKGLFLLMGAKMHWTSVVAGKGWGQAKPVERAFGVGGLEEYVDKHPALAGAYTGPNPQAKPDNYGDRAVDAELFLKTLAEGVAMFNARTGRETEMCGGKLSFDDVFEREYARTIVRKPTEEQKRMLLLPAEAVNVSRKGEFALKVGGSLKGAKNVYYNMALMNAGVKKVVVRFDPQQLHSTVYCYTLDGRFICEAECLAPVAFNDAAAGREYRRRQKQLKSATKAAIKAQKQMDALEVAELLPQIAEPEAPESRIVGIFRPSGNTERVKNQERDDEYETERDEYLNHSLDILEQNRRKKAI

SEQ ID NO: 3 - protein, 459 aa, Vibrio harveyi
MTHSDAKLWAQEQFGQAQLKDPRRTQRLISLATSIANQPGVSVAKLPFSPADMEGAYRFIRNENINAEDIAEAGFQSTVSRANEHKELLALEDTTTLSFPHRSIKEELGHTNQGDRTRALHVHSTLLFAPQSQTIVGLIEQQRWSRDITKRGQKHQHATRPYKEKESYKWEQASRRVVERLGDKMLDVISVCDREADLFEYLTYKRQHQQRFVVRSMQSRCLEEHAQKLYDYAQALPSVETKALTIPQKGGRKARNVKLDVKYGQVTLKAPANKKEHAGIPVYYVGCLEQGTSKDKLAWHLLTSEPINNVDDAMRIIGYYERRWLIEDFHKVWKSEGTDVESLRLQSKDNLERLSVIYAFVATRLLALRFMKEVDELTKESCEKVLGQKAWKLLWLKLESKTLPKEVPDMGWAYKNLAKLGGWKDTKRTGRASIKVLWEGWFKLQTILEGYELAMSLDH

SEQ ID NO: 4 - protein, 273 aa, Escherichia coli
MAKANSSFSEVQIARRIKEGRGQGHGKDYIPWLTVQEVPSSGRSHRIYSHKTGRVHHLLSDLELAVFLSLEWESSVLDIREQFPLLPSDTRQIAIDSGIKHPVIRGVDQVMSTDFLVDCKDGPFEQFAIQVKPAAALQDERTLEKLELERRYWQQKQIPWFIFTDKEINPVVKENIEWLYSVKTEEVSAELLAQLSPLAHILQEKGDENIINVCKQVDIAYDLELGKTLSEIRALTANGFIKFNIYKSFRANKCADLCISQVVNMEELRYVAN

SEQ ID NO: 5 - protein, 340 aa, Synthetic construct
MGKSKEISQDLRKKIVDLHKSGSSLGAISKRLKVPRSSVQTIVRKYKHHGTTQPSYRSGRRRVLSPRDERTLVRKVQINPRTTAKDLVKMLEETGTKVSISTVKRVLYRHNLKGRSARKKPLLQNRHKKARLRFATAHGDKDRTFWRNVLWSDETKIELFGHNDHRYVWRKKGEACKPKNTIPTVKHGGGSIMLWCGFAAGGTGALHKIDGIMRKENYVDILKQHLKTSVRKLKLGRKWVFQMDNDPKHTSKVVAKWLKDNKVKVLEWPSQSPDLNPIENLWAELKKRVRARRPTNLTQLHQLCQEEWAKIHPTYCGKLVEGYPKRLTQVKQFKGNATKY

SEQ ID NO: 6 - DNA, 19 bp, Synthetic construct
  source 1..19: mol_type = other DNA
ctgtctcttatacacatct

SEQ ID NO: 7 - DNA, 19 bp, Synthetic construct
  source 1..19: mol_type = other DNA
agatgtgtataagagacag

SEQ ID NO: 8 - skipped

SEQ ID NO: 9 - DNA, 27 bp, synthetic construct
  source 1..27: mol_type = other DNA
ctgtctcttatacacatctctaggctc

(19) United States
(12) Patent Application Publication
(10) Pub. No.: US 2025/0066770 A1
(43) Pub. Date: Feb. 27, 2025

(54) MODIFIED TRANSPOSONS, COMPOSITIONS AND USES THEREOF

(71) Applicant: 10x Genomics, Inc., Pleasanton, CA (US)

(72) Inventor: Justin Costa, Union City, CA (US)

(21) Appl. No.: 18/948,720

(22) Filed: Nov. 15, 2024

Related U.S. Application Data
(63) Continuation of application No. PCT/US2023/067070, filed on May 16, 2023.
(60) Provisional application No. 63/342,845, filed on May 17, 2022.

(51) Int. Cl. C12N 15/10

(52) U.S. Cl. CPC C12N 15/1082; C12N 15/1065

(57) ABSTRACT

Disclosed herein are modified split mirrored transposons or double-stranded split mirrored transposon (SMT) compositions and methods of using SMTs. The methods include producing SMTs and using SMTs for nucleic acid analysis.

### CROSS-REFERENCE TO RELATED APPLICATIONS

Pursuant to 35 U.S.C. § 119(e), this application is a continuation of International Application PCT/US2023/067070, with an international filing date of May 16, 2023, which claims the benefit of U.S. Provisional Application Ser. No. 63/342,845, filed May 17, 2022, the entire contents of which is incorporated by reference herein.

### SEQUENCE LISTING

This application contains a Sequence Listing that has been submitted electronically as an XML file named “47706-0325001_SL_ST26.XML.” The XML file, created on Nov. 12, 2024, is 10,335 bytes in size. The material in the XML file is hereby incorporated by reference in its entirety.

### BACKGROUND

Transpositional systems have been used successfully as a powerful tool for introducing non-native sequences into a target nucleic acid of interest. A transposome includes a transposase enzyme and transposon sequences, and the transposon sequences being specific to a particular transposase. However, additional sequences can be appended to a transposon sequence such that those additional sequences are also inserted into the resultant fragmented and tagged target nucleic acid upon transposition. The transpositional method can then be used as a tool to generate nucleic acid libraries of fragmented and tagged molecules for use in, for example, next generation sequencing methods or their use in assays directed to query accessible chromatic across a genome, such as ATAC-seq methodologies.

However, it would be useful if transposition could also allow for tracking of associations between the fragmented and tagged nucleic acids, thereby identifying contiguity of a nucleic acid. As such, described herein are methods and compositions for artificial sequences in conjunction with transposon sequences and their uses in methods of preparing libraries of tagged nucleic acid fragments.

### SUMMARY

Transpositional systems are useful for introducing non-native sequences into a target cell of interest and, in some cases, can fragment the nucleic acid into which the non-native sequence can be inserted. However, it would be useful if associations between the fragmented and tagged nucleic acids could be tracked, thereby identifying contiguity of a nucleic acid after fragmentation. The present disclosure generally describes compositions and methods for making and using modified transposons. In some cases, the compositions and methods described herein can identify contiguity of a nucleic acid following fragmentation. For example, “contiguity” of a nucleic acid can mean the ability to reassemble which fragments had been contiguous nucleic acid sequences before being fragmented.

Provided herein are double-stranded transposon nucleic acid compositions comprising (a) a restriction enzyme site sequence flanked by first and second hairpin sequences; (b) a first molecular identifier sequence and a second molecular identifier sequence that flank the first and second hairpin sequences; and (c) a first mosaic end sequence and a second mosaic end sequence that flank the first and second molecular identifier sequences.

In some cases, the double-stranded transposon nucleic acid composition further comprising the first mosaic end and the second mosaic end bound to transposase enzymes.

In some cases, the transposase enzyme is a Tn5 transposase enzyme, a Mu transposase enzyme, a Tn7 transposase enzyme, a Vibhar transposase enzyme, a Mariner transposase enzyme, or functional derivatives thereof. In some cases, the transposase enzyme is Tn5. In some cases, the Tn5 comprises a sequence that has at least about 70%, 75%, 80%, 85%, 90%, or 95% sequence identity to SEQ ID NO: 1. In some cases, the Tn5 comprises SEQ ID NO: 1.

In some cases, the first molecular identifier sequence and the second molecular identifier sequence are the same sequences, or complements thereof. In some cases, the first molecular identifier sequence and the second molecular identifier sequence are unique for each double-stranded transposon nucleic acid composition. In some cases, the first molecular identifier sequence and the second molecular identifier sequence each comprise about 10 to about 20 nucleotides. In some cases, the composition is synthetically produced.

Also provided herein are compositions for a transposome complex comprising (a) one or more transposase enzymes; (b) a transposon sequence, wherein the transposon sequence comprises a unique restriction enzyme site flanked by a first and second hairpin sequence, wherein the first hairpin sequence is complementary to the second hairpin sequence, wherein the first and second hairpin sequences are flanked by a first and a second molecular identifier sequence, wherein the first molecular identifier sequence is complementary to the second molecular identifier sequence, wherein the first and second molecular identifier sequences are flanked by a first and second transposase recognition sequence; and (c) a transposase enzyme bound by the first transposase recognition sequence a transposase enzyme bound by the second transposase recognition sequence.

In some cases, the transposase enzyme is a Tn5 transposase enzyme, a Mu transposase enzyme, a Tn7 transposase enzyme, a Vibhar transposase enzyme, a Mariner transposase enzyme, or functional derivatives thereof. In some cases, the transposase enzyme is Tn5. In some cases, the Tn5 comprises a sequence that has at least about 70%, 75%, 80%, 85%, 90%, or 95% sequence identity to SEQ ID NO: 1. In some cases, the Tn5 comprises SEQ ID NO: 1.

In some cases, the transposome complex is one complex in a plurality of transposome complexes, wherein each transposome complex comprises a different molecular identifier sequence and its complement. In some cases, the first molecular identifier sequence and the second molecular identifier sequence are the same sequences, or complements thereof. In some cases, the first molecular identifier sequence and the second molecular identifier sequence are unique for each transposome complex. In some cases, the first molecular identifier sequence and the second molecular identifier sequence each comprise about 10 to about 20 nucleotides. In some cases, the composition is synthetically produced.

Also provided herein are methods of producing a transposome complex, the method comprising (a) providing an oligonucleotide sequence comprising: (i) a first restriction enzyme site sequence, (ii) a second restriction enzyme site sequence, (iii) a molecular identifier sequence, and (iv) a first and a second hairpin sequence that flank a third restriction enzyme site, wherein the two hairpin sequences are substantially complementary to each other; (b) hybridizing the first and the second hairpin sequence together, thereby generating a hairpin loop; (c) extending the hairpin loop to generate a double-stranded sequence comprising the molecular identifier and its complement, the first restriction enzyme site sequence and its complement, and the second restriction enzyme site and its complement; (d) hybridizing a primer comprising a mosaic end at its 5′ end to the 3′ overhang; (e) generating a complete double-stranded nucleic acid molecule using a strand displacing enzyme thereby relieving the hairpin loop structure; (f) digesting the double-stranded nucleic acid molecule with a second restriction enzyme that generates a nick at one end of the molecule and generates a 3′ overhang on the other end; (g) ligating a duplexed mosaic end to the double-stranded sequence, thereby generating a double-stranded transposon nucleic acid comprising: i) two mosaic ends, ii) two molecular identifier sequences, and iii) a nucleic acid sequence comprising the two hairpin sequences separated by a third restriction enzyme site; and (h) adding one or more transposase enzymes that bind to mosaic ends to generate a transposome complex.

In some cases, the first restriction enzyme site is recognized by a first nicking enzyme. In some cases, the second restriction enzyme site is recognized by a second nicking enzyme. In some cases, the first nicking enzyme and the second nicking enzyme are different.

Also disclosed herein are methods of producing a plurality of tagmented nucleic acid molecules, the method comprising (a) permeabilizing a biological sample under conditions sufficient to make a nucleic acid molecule in the biological sample accessible to transposon insertion; (b) providing the composition of any one of claims 1-10 and a transposase enzyme to the biological sample under conditions wherein the composition is inserted into the nucleic acid molecule, thereby generating a tagmented nucleic acid molecule; and (c) collecting the tagmented nucleic acid molecule. In some cases, step (c) further comprises analyzing the tagmented nucleic acid molecule and correlating its presence in the biological sample.

In some cases, the biological sample comprises one or more single cells. In some cases, the single cells are separated by one or more partitions. In some cases, the analyzing comprises determining all or a portion of a sequence of the tagmented nucleic acid molecule, or a complement thereof, and using the determined sequences to determine the identity and/or abundance of a nucleic acid molecule from the biological sample. In some cases, the determining all or a portion of a sequence of the tagmented nucleic acid molecule comprises high-throughput sequencing. In some cases the nucleic acid molecule is RNA. In some cases, the nucleic acid molecule is mRNA. In some cases, the nucleic acid molecule is DNA. In some cases, the nucleic acid molecule is genomic DNA.

In some cases, the method further comprises, before step (a), placing the biological sample on an array comprising a plurality of capture probes, wherein a capture probe of the plurality of capture probes comprises: (i) a spatial barcode and (ii) a capture domain. In some cases, the capture probe further comprises a cleavage domain, one or more functional domains, a molecular identifier sequence, or combinations thereof. In some cases, the method further comprises before step (b), hybridizing the tagmented nucleic acid molecule to the capture probe; and extending the capture probe using the tagmented nucleic acid molecule as a template, there by generating an extended capture probe and an extended tagmented nucleic acid molecule.

In some cases, the extending utilizes a polymerase, optionally wherein the polymerase comprises strand displacement activity. In some cases, the analyzing further comprises determining (i) a sequence of the spatial barcode or a complement thereof, and (ii) all or a portion of a sequence of the tagmented nucleic acid molecule, or a complement thereof, and using the determined sequences of (i) and (ii) to determine the abundance and the location of the nucleic acid molecule in the biological sample. In some cases, the nucleic acid molecule is RNA. In some cases, the nucleic acid molecule is mRNA. In some cases, the nucleic acid molecule is genomic DNA.

In some cases, the nucleic acid molecule is genomic DNA. In some cases, the method further comprises before step (a), placing the biological sample on an array comprising a plurality of capture probes, wherein a capture probe of the plurality of capture probes comprises: (i) a spatial barcode and (ii) a capture domain. In some cases, the capture probe further comprises a cleavage domain, one or more functional domains, a molecular identifier sequence, or combinations thereof. In some cases, step (c) further comprises generating a tagmented genomic DNA. In some cases, step (d) further comprises binding the tagmented genomic DNA to the capture probe. In some cases, the binding comprises hybridizing a splint oligonucleotide, or a portion thereof, to the capture domain, or a portion thereof, of the capture probe and to a portion of the tagmented genomic DNA. In some cases, the analyzing comprises determining (i) a sequence of the spatial barcode or a complement thereof, and (ii) all or a portion of a sequence of the tagmented genomic DNA, or a complement thereof, and using the determined sequences of (i) and (ii) to determine the abundance and the location of the genomic DNA in the biological sample.

In some cases, any of the methods described herein further comprise extending a 3′ end of the capture probe using the tagmented genomic DNA as a template. In some cases, the extending is performed using a DNA polymerase having strand displacement activity.

In any of the methods described herein, the permeabilizing the biological sample uses chemical permeabilization, an enzymatic permeabilization, or both.

In some cases, the method further comprises before step (a) mounting the biological sample on a first substrate. In some cases, the method further comprises aligning the first substrate with a second substrate comprising an array, such that at least a portion of the biological sample is aligned with at least a portion of the array, wherein the array comprises a plurality of capture probes, wherein a first capture probe of the plurality of capture probes comprises: (i) a first spatial barcode and (ii) a first capture domain.

Also described herein is are kits comprising: (a) any of the transposome complexes described herein; (b) one or more of a DNA polymerase, a ligase, and a reverse transcriptase; and (c) instructions for generating tagmented nucleic acid molecules.

Also provided herein are double-stranded split mirrored transposon nucleic acid compositions (also called double-stranded transposon nucleic acid compositions) including (a) a plurality of restriction enzyme sites comprising a first restriction enzyme site, a second restriction enzyme site, and a third restriction enzyme site, wherein the third restriction enzyme site is flanked by sequences that are complementary to one another and that are capable of forming a hairpin; (b) a first molecular identifier sequence and a second molecular identifier sequence; and (c) a first mosaic end sequence and a second mosaic end sequence.

In some cases, any of the compositions provided herein further include a transposase enzyme affixed to each of the first mosaic end sequence and the second mosaic end sequence. In some cases, the transposase enzyme is a Tn5 transposase enzyme, a Mu transposase enzyme, a Tn7 transposase enzyme, a Vibhar transposase enzyme (e.g., a Vibrio harveyi transposase enzyme), a Mariner transposase enzyme, or functional derivatives thereof. In some cases, the transposase enzyme is Tn5. Exemplary Tn5 transposase enzymes can be Escherichia coli Tn5 transposases, such as the Tn5 transposase of SEQ ID NO: 1. In some cases, the Tn5 transposase enzyme comprises a sequence that has at least about 70%, 75%, 80%, 85%, 90%, or 95% sequence identity to SEQ ID NO: 1. In some cases, the Tn5 comprises SEQ ID NO: 1. In some cases, the Tn5 transposase enzyme comprises a sequence that has 0, 1, 2, 3, 4, 5, 6, 7, 8, 9, or 10 point mutations compared to SEQ ID NO: 1.

In some cases the composition comprises, in order, a first mosaic end, the first molecular identifier sequence, a third restriction enzyme site, the second molecular identifier sequence, and a second mosaic end. In some cases, the first molecular identifier sequence and the second molecular identifier sequence in a split transposon sequence are identical sequences. In some cases, the first molecular identifier sequence and the second molecular identifier sequence each comprise about 10 to about 20 nucleotides. In some cases, the first molecular identifier sequence and the second molecular identifier sequence each comprise about 15 nucleotides.

In some cases, the composition is synthetically produced.

In some cases, the first restriction enzyme site is recognized by a first nicking enzyme. In some cases, the second restriction enzyme site is recognized by a second nicking enzyme. In some cases, the first nicking enzyme and the second nicking enzyme are different.

Also provided herein are methods of preparing a library of analytes from a biological sample including permeabilizing the biological sample under conditions sufficient to make an analyte of the analytes in the biological sample accessible to transposon insertion; providing any of the compositions described herein and a transposase enzyme to the biological sample under conditions wherein the composition is inserted into the analyte; allowing the transposase enzyme to excise the inserted transposon sequence from the analyte, thereby generating a fragmented analyte; and collecting the fragmented analyte.

In some cases, the permeabilizing the biological sample uses chemical permeabilization, an enzymatic permeabilization, or both. In some cases, any of the methods described herein further include determining all or a portion of a sequence of the fragmented analyte, or a complement thereof, and using the determined sequences to determine the identity and/or abundance of the analytes from the biological sample. In some cases, the determining all or a portion of a sequence of the fragmented analyte comprises high-throughput sequencing.

Also provided herein are methods of analyzing an analyte present in a biological sample including permeabilizing the biological sample under conditions sufficient to make the analyte in the biological sample accessible to transposon insertion; providing any of the compositions described herein and a transposase enzyme to the biological sample under conditions wherein the composition is inserted into the analyte; allowing the transposase enzyme to excise the inserted transposon sequence from the analyte, thereby generating a fragmented analyte; and analyzing the fragmented analyte, thereby analyzing the analyte present in the biological sample.

In some cases, the permeabilizing the biological sample uses chemical permeabilization, an enzymatic permeabilization, or both. In some cases, the analyzing the fragmented analyte further comprises determining all or a portion of a sequence of the fragmented analyte, or a complement thereof, and using the determined sequences to determine the identity and/or abundance of the analytes from the biological sample. In some cases, the determining all or a portion of a sequence of the fragmented analyte comprises using high-throughput sequencing.

Also provided herein are methods of analyzing an analyte present in a single cell biological sample including permeabilizing the single cell biological sample under conditions sufficient to make the analyte in the single cell biological sample accessible to transposon insertion; providing any of the compositions described herein and a transposase enzyme to the single cell biological sample under conditions wherein the composition is inserted into the analyte; allowing the transposase enzyme to excise the inserted transposon sequence from the analyte, thereby generating a fragmented analyte; and analyzing the fragmented analyte, thereby analyzing the analyte present in the single cell biological sample.

In some cases, the permeabilizing the single cell biological sample uses chemical permeabilization, an enzymatic permeabilization, or both. In some cases, the analyzing the fragmented analyte further comprises determining all or a portion of a sequence of the fragmented analyte, or a complement thereof, and using the determined sequences to determine the identity and/or abundance of the analytes from the biological sample. In some cases, the determining all or a portion of a sequence of the fragmented analyte comprises using high-throughput sequencing.

In any of the methods described herein, the analyte is RNA. In any of the methods described herein, the analyte is mRNA. In any of the methods described herein, the analyte is DNA. In any of the methods described herein, the analyte is genomic DNA (gDNA). In any of the methods described herein, the analyte is complementary DNA (cDNA).

Also described herein are methods of enhancing detection of abundance and location of an analyte in a biological sample including (a) placing the biological sample on an array comprising a plurality of capture probes, wherein a capture probe of the plurality of capture probes comprises: (i) a spatial barcode and (ii) a capture domain; (b) hybridizing the analyte to the capture probe; (c) extending the capture probe using the analyte as a template, there by generating an extended capture probe; (d) providing to the array a plurality of double-stranded split mirrored transposon nucleic acid compositions, wherein a double-stranded split mirrored transposon nucleic acid composition of the plurality includes: (i) a plurality of restriction enzyme sites comprising a first restriction enzyme site, a second restriction enzyme site, and a third restriction enzyme site, wherein the third restriction enzyme site is flanked by sequences that are complementary to one another and that are capable of forming a hairpin; (ii) a first molecular identifier sequence and a second molecular identifier sequence; (iii) a first mosaic end sequence and a second mosaic end sequence; and (iv) a transposase enzyme; (e) integrating the double-stranded split mirrored transposon nucleic acid composition into the extended capture probe or a complement thereof; and (f) determining (i) a sequence of the spatial barcode or a complement thereof, and (ii) all or a portion of a sequence of the analyte, or a complement thereof, and using the determined sequences of (i) and (ii) to enhance determination of the abundance and the location of the analyte in the biological sample compared to a method that does not utilize the plurality of double-stranded split mirrored transposon nucleic acid compositions.

In some cases, the extending the capture probe utilizes a polymerase, optionally wherein the polymerase comprises strand displacement activity.

Also provided herein are methods for determining abundance and location of accessible genomic DNA in a biological sample including (a) placing the biological sample on an array comprising a plurality of capture probes, wherein a capture probe of the plurality of capture probes comprises: (i) a spatial barcode and (ii) a capture domain; (b) providing to the biological sample a plurality of double-stranded split mirrored transposon nucleic acid compositions, wherein a double-stranded split mirrored transposon nucleic acid composition of the plurality includes (i) a plurality of restriction enzyme sites comprising a first restriction enzyme site, a second restriction enzyme site, and a third restriction enzyme site, and wherein the third restriction enzyme site is flanked by sequences that are complementary to one another and that are capable of forming a hairpin; (ii) a first molecular identifier sequence and a second molecular identifier sequence; (iii) a first mosaic end sequence and a second mosaic end sequence; and (iv) a transposase enzyme; (c) integrating the double-stranded split mirrored transposon nucleic acid composition into accessible genomic DNA, thereby generating fragmented genomic DNA; (d) binding the fragmented genomic DNA to the capture probe; and (e) determining (i) a sequence of the spatial barcode or a complement thereof, and (ii) all or a portion of a sequence of the fragmented genomic DNA, or a complement thereof, and using the determined sequences of (i) and (ii) to determine the abundance and the location of the accessible genomic DNA in the biological sample.

In any of the methods described herein, the analyte is RNA. In any of the methods described herein, the analyte is mRNA. In any of the methods described herein, the analyte is DNA. In any of the methods described herein, the analyte is genomic DNA (gDNA).

In some cases, the double-stranded split mirrored transposon nucleic acid composition is synthetically produced. In some cases, the first restriction enzyme site is recognized by a first nicking enzyme. In some cases, the second restriction enzyme site is recognized by a second nicking enzyme. In some cases, the first nicking enzyme and the second nicking enzyme are different. In some cases, the first restriction enzyme site is six nucleotides in length. In some cases, the second restriction enzyme site is six nucleotides in length.

In some cases, the molecular identifier sequence is about 10 to about 20 nucleotides in length. In some cases, the molecular identifier sequence is about 15 nucleotides in length.

In some cases, the transposase enzyme is a Tn5 transposase enzyme, a Mu transposase enzyme, a Tn7 transposase enzyme, a Vibhar transposase enzyme (e.g., a Vibrio harveyi transposase enzyme), a Mariner transposase enzyme, or functional derivatives thereof. In some cases, the transposase enzyme is Tn5. In some cases, the Tn5 comprises a sequence that has at least about 70%, 75%, 80%, 85%, 90%, or 95% sequence identity to SEQ ID NO: 1. In some cases, the Tn5 comprises a sequence comprising SEQ ID NO: 1.

In some cases, the array comprises one or more features. In some cases, the one or more features comprises a bead.

In some cases, the capture probe further comprises a cleavage domain, one or more functional domains, a molecular identifier sequence, or combinations thereof.

In some cases, the binding in step (d) comprises hybridizing the splint oligonucleotide, or a portion thereof, to the capture domain, or a portion thereof, of the capture probe. In some cases, the binding in step (d) comprises hybridizing the splint oligonucleotide, or a portion thereof, to a transposon end sequence or a portion thereof.

In some cases, the method further includes extending a 3′ end of the capture probe using the fragmented genomic DNA as a template. In some cases, the extending step is performed using a DNA polymerase having strand displacement activity.

In some cases, the determining comprises sequencing (i) the sequence of the spatial barcode or a complement thereof, and (ii) all or a portion of the sequence of the analyte or the fragmented genomic DNA or a complement thereof.

In some cases, any of the methods described herein further include imaging the biological sample before or after contacting the biological sample with the array. In some cases, any of the methods described heron further include staining the biological sample. In some cases, the staining comprises hematoxylin and eosin (H&E) staining.

In some cases, the providing to the biological sample the plurality of double-stranded split mirrored transposon nucleic acid compositions is performed under a chemical permeabilization condition, under an enzymatic permeabilization condition, or both. In some cases, the providing to the biological sample the plurality of double-stranded split mirrored transposon nucleic acid compositions is performed after an enzymatic pre-permeabilization condition. In some cases, the enzymatic pre-permeabilization condition comprises a protease. In some cases, the protease is a pepsin, a collagenase, proteinase K, and combinations thereof.

Also provided herein are kits for practicing the method of preparing a library of analytes from a biological sample including: (a) a double-stranded split mirrored transposon nucleic acid composition including: (i) a plurality of restriction enzyme sites comprising a first restriction enzyme site, a second restriction enzyme site, and a third restriction enzyme site, wherein the third restriction enzyme site is unique to a target genome, and wherein the third restriction enzyme site is flanked by sequences that are complementary to one another and that are capable of forming a hairpin; (ii) a first molecular identifier (MI) and a second MI; (iii) a first mosaic end sequence and a second mosaic end sequence; and (iv) a transposase enzyme; (b) an array comprising a plurality of capture probes, wherein a capture probe of the plurality of capture probes comprises: (i) a spatial barcode and (ii) a capture domain; (c) one or more restriction enzymes; (d) one or more enzymes selected from a polymerase, a ligase, and a reverse transcriptase; and (e) instructions for performing any of the methods described herein.

Also provided herein are kits for analyzing an analyte present in a biological sample including: (a) a double-stranded split mirrored transposon nucleic acid composition including: (i) a plurality of restriction enzyme sites comprising a first restriction enzyme site, a second restriction enzyme site, and a third restriction enzyme site, and wherein the third restriction enzyme site is flanked by sequences that are complementary to one another and that are capable of forming a hairpin; (ii) a first molecular identifier (MI) and a second MI; (iii) a first mosaic end sequence and a second mosaic end sequence; and (iv) a transposase enzyme; (b) an array comprising a plurality of capture probes, wherein a capture probe of the plurality of capture probes comprises: (i) a spatial barcode and (ii) a capture domain; (c) one or more restriction enzymes; (d) one or more enzymes selected from a polymerase, a ligase, and a reverse transcriptase; and (e) instructions for performing any of the methods described herein.

Also provided herein are kits for practicing the method of analyzing an analyte present in a single cell biological sample including: (a) a double-stranded split mirrored transposon nucleic acid composition including: (i) a plurality of restriction enzyme sites comprising a first restriction enzyme site, a second restriction enzyme site, and a third restriction enzyme site, wherein the third restriction enzyme site is unique to a target genome, and wherein the third restriction enzyme site is flanked by sequences that are complementary to one another and that are capable of forming a hairpin; (ii) a first molecular identifier (MI) and a second MI; (iii) a first mosaic end sequence and a second mosaic end sequence; and (iv) a transposase enzyme; (b) an array comprising a plurality of capture probes, wherein a capture probe of the plurality of capture probes comprises: (i) a spatial barcode and (ii) a capture domain; (c) one or more restriction enzymes; (d) one or more enzymes selected from a polymerase, a ligase, and a reverse transcriptase; and (e) instructions for performing the method of any one of claims 23-30

Also provided herein are kits for enhancing detection of abundance and location of an analyte in a biological sample including: (a) a double-stranded split mirrored transposon nucleic acid composition including: (i) a plurality of restriction enzyme sites comprising a first restriction enzyme site, a second restriction enzyme site, and a third restriction enzyme site, and wherein the third restriction enzyme site is flanked by sequences that are complementary to one another and that are capable of forming a hairpin;(ii) a first molecular identifier (MI) and a second MI; (iii) a first mosaic end sequence and a second mosaic end sequence; and (iv) a transposase enzyme; (b) an array comprising a plurality of capture probes, wherein a capture probe of the plurality of capture probes comprises: (i) a spatial barcode and (ii) a capture domain; (c) one or more restriction enzymes; (d) one or more enzymes selected from a polymerase, a ligase, and a reverse transcriptase; and (e) instructions for performing any of the methods described herein.

Also provided herein are kits for determining abundance and location of accessible genomic DNA in a biological sample including: (a) a double-stranded split mirrored transposon nucleic acid composition including:(i) a plurality of restriction enzyme sites comprising a first restriction enzyme site, a second restriction enzyme site, and a third restriction enzyme site, and wherein the third restriction enzyme site is flanked by sequences that are complementary to one another and that are capable of forming a hairpin; (ii) a first molecular identifier (MI) and a second MI; (iii) a first mosaic end sequence and a second mosaic end sequence; and (iv) a transposase enzyme; (b) an array comprising a plurality of capture probes, wherein a capture probe of the plurality of capture probes comprises: (i) a spatial barcode and (ii) a capture domain; (c) one or more restriction enzymes; (d) one or more enzymes selected from a polymerase, a ligase, and a reverse transcriptase; and (e) instructions for performing any of the methods described herein.

All publications, patents, and patent applications mentioned in this specification are herein incorporated by reference to the same extent as if each individual publication, patent, patent application, or item of information was specifically and individually indicated to be incorporated by reference. To the extent publications, patents, patent applications, and items of information incorporated by reference contradict the disclosure contained in the specification, the specification is intended to supersede and/or take precedence over any such contradictory material.

Where values are described in terms of ranges, it should be understood that the description includes the disclosure of all possible sub-ranges within such ranges, as well as specific numerical values that fall within such ranges irrespective of whether a specific numerical value or specific sub-range is expressly stated.

The term “about” or “approximately” as used herein means within an acceptable error range for the particular value as determined by one of ordinary skill in the art, which will depend in part on how the value is measured or determined, i.e., the limitations of the measurement system. For example, “about” can mean within an acceptable standard deviation, per the practice in the art. Alternatively, “about” can mean a range of up to ±20%, preferably up to ±10%, more preferably up to ±5%, and more preferably still up to ±1% of a given value. Alternatively, particularly with respect to biological systems or processes, the term can mean within an order of magnitude, preferably within 2-fold, of a value. Where particular values are described in the application and claims, unless otherwise stated, the term “about” is implicit and in this context means within an acceptable error range for the particular value.

The term “substantially complementary” or “substantially hybridize” used herein means that a first sequence is at least 50%, 55%, 60%, 65%, 70%, 75%, 80%, 85%, 90%, 95%, 97%, 98% or 99% identical to the complement of a second sequence over a region of 2, 3, 4, 5, 6, 7, 8, 9, 10, 11, 12, 13, 14, 15, 16, 17, 18, 19, 20-40, 40-60, 60-100, or more nucleotides, or that the two sequences hybridize under stringent hybridization conditions. Substantially complementary also means that a sequence in one strand is not completely and/or perfectly complementary to a sequence in an opposing strand, but that sufficient bonding occurs between bases on the two strands to form a stable hybrid complex in set of hybridization conditions (e.g., salt concentration and temperature). Such conditions can be predicted by using the sequences and standard mathematical calculations known to those skilled in the art.

The term “each,” when used in reference to a collection of items, is intended to identify an individual item in the collection but does not necessarily refer to every item in the collection, unless expressly stated otherwise, or unless the context of the usage clearly indicates otherwise.

Various embodiments of the features of this disclosure are described herein. However, it should be understood that such embodiments are provided merely by way of example, and numerous variations, changes, and substitutions can occur to those skilled in the art without departing from the scope of this disclosure. It should also be understood that various alternatives to the specific embodiments described herein are also within the scope of this disclosure.

### DESCRIPTION OF DRAWINGS

The following drawings illustrate certain embodiments of the features and advantages of this disclosure. These embodiments are not intended to limit the scope of the appended claims in any manner. Like reference symbols in the drawings indicate like elements.

FIG. 1 shows an exemplary oligonucleotide sequence with a first restriction enzyme site sequence (Nt.BspQI, underlined), a second restriction enzyme site (Nb.BsrDI, underlined), a molecular identifier (MI; SEQ ID NO: 8) sequence, a first hairpin sequence (hairpin, italicized and underlined) and a second hairpin sequence (hairpin′, italicized and underlined), and a third restriction enzyme site (Srf1, underlined).

FIG. 2 shows an exemplary oligonucleotide sequence with the first hairpin sequence and second hairpin sequence hybridized to each other.

FIG. 3 shows an exemplary double stranded hairpin oligonucleotide comprising an extended sequence 3′ of the second hairpin′ sequence.

FIG. 4 shows an exemplary hairpin oligonucleotide of FIG. 3 where a nicking endonuclease (Nt.BspQI) is used to generate a 3′ overhang.

FIG. 5 shows an exemplary hairpin oligonucleotide of FIG. 4 onto which a primer (SEQ ID NO: 9) with a mosaic end 1 (italicized; SEQ ID NO: 6) is annealed to the 3′ overhang, leaving a gap and generating a 5′ overhang comprising the mosaic end.

FIG. 6 shows an exemplary double stranded oligonucleotide with the 3′ end extended to generate a compliment of the 5′ mosaic end overhang (mosaic end 2; SEQ ID NO: 7) of FIG. 5, the gap is extended and the hairpin is released using, for example, a strand displacing polymerase.

FIG. 7 shows an exemplary double stranded oligonucleotide of FIG. 6 that is digested with a second nicking endonuclease Nb.BsrDI that cleaves at two places in the oligonucleotide (arrows) to generate a 3′ overhang on one end and a nick on the opposite end of the oligonucleotide.

FIG. 8 shows an exemplary double stranded oligonucleotide of FIG. 7 wherein a sticky end double stranded adaptor comprising the mosaic end sequence is ligated to the 3′ overhang, the nick on the opposite end is sealed, thereby generating a double-stranded modified transposon, also referred to as a double-stranded transposon nucleic acid or a split mirrored transposon, with a third restriction enzyme site Srf1 flanked by molecular identifier sequences (MIs; SEQ ID NO: 8) and mosaic ends located at the 3′ and 5′ ends.

FIG. 9 shows an exemplary modified or split mirrored transposon (SMT) complexed with transposases, thereby generating a transposome complex.

FIGS. 10A-B show an exemplary workflow of processing a nucleic acid with split mirrored transposons of FIG. 9. FIG. 10A shows an exemplary strand of nucleic acid with four (labeled A, B, C, D) inserted SMTs. FIG. 10B shows exemplary tagmented nucleic acid products, after tagmentation by the SMT complexes and digestion with Srf1 and optionally following ligation of adaptor sequences.

FIG. 11 shows an exemplary workflow of tagmenting nucleic acids on a substrate such as a spatial array using a SMT complex in a transpositional system.

### DETAILED DESCRIPTION

The present disclosure generally describes compositions and methods for making and using modified, or split-mirrored, transposons that can be complexed with transposases thereby generating a split mirrored transposon (SMT) transpositional system. Nucleic acids such as DNA or RNA contain extensive biological information. Some sequencing technologies are only able to sequence short nucleic acid sequences (e.g. <1000-1500 bp), for example, because of library preparation limitations or sequencing technology limitations. Split mirrored transposons (SMTs) can provide long read sequence information using standard sequencing techniques by providing a molecular identifier (MI) sequence to mark nucleic acid ends that would have been connected in the cell or tissue. SMTs are inserted into nucleic acids and contain MIs that are split when the transposase cuts and inserts the SMTs into the nucleic acid sequences. Long sequences can then be reconstructed by matching the MIs computationally from the sequencing data.

The methods and compositions described herein can be utilized to study various types of cancers. For example, chromothripsis is a process that some cancers undergo, wherein a single chromosome brakes into, for example, thousands of fragments that the cell tries to reassemble, leading to multiple issues such as chromosomal deletions, translocations, inversions, aberrant fusions, and the like. The disclosed transpositional methods would find utility in identifying these cancer cell processes. Further, the disclosed transpositional methods can also be used to study do novo genome assembly. For example, in microbiology there are certain bacterial species that are difficult if not impossible to culture and the genomic information of such bacterial species is limited or absent. Practicing the present methods and compositions on nucleic acids extracted from such bacterial species would provide genomic information that is not available practicing other methods.

Use of SMTs can be useful for many different applications. For example, SMTs can be used to determine contiguity information of target nucleic acid sequences (See, for example, Patent Nos: EP 3207134 and EP 2527438, each of which is incorporate in its entirety herein). In another example, SMTs can be combined with spatial gene expression analysis, or spatial transcriptomics methodologies and compositions, to provide a vast amount of gene expression data from a biological sample at high spatial resolution, while retaining native genomic and spatial context. Spatial analysis methods and compositions can include, e.g., the use of a capture probe including a spatial barcode (e.g., a nucleic acid sequence that provides information as to the location or position of an analyte within a cell or a tissue sample (e.g., mammalian cell or a mammalian tissue sample) and a capture domain that is capable of binding to an analyte (e.g., a protein related nucleic acid and/or a DNA or RNA) produced by and/or present in a cell.

Some general terminology that may be used in this disclosure can be found in Section (I)(b) of WO 2020/176788 and/or U.S. Patent Application Publication No. 2020/0277663. Typically, a “barcode” is a label, or identifier, that conveys or is capable of conveying information (e.g., information about an analyte in a sample, a bead, and/or a capture probe). A barcode can be part of an analyte, or independent of an analyte. A barcode can be attached to an analyte. A particular barcode can be unique relative to other barcodes. For the purpose of this disclosure, an “analyte” can include any biological substance, structure, moiety, or component to be analyzed. The term “target” can similarly refer to an analyte of interest.

Analytes can be broadly classified into one of two groups: nucleic acid analytes, and non-nucleic acid analytes. Examples of non-nucleic acid analytes include, but are not limited to, lipids, carbohydrates, peptides, proteins, glycoproteins (N-linked or O-linked), lipoproteins, phosphoproteins, specific phosphorylated or acetylated variants of proteins, amidation variants of proteins, hydroxylation variants of proteins, methylation variants of proteins, ubiquitylation variants of proteins, sulfation variants of proteins, viral proteins (e.g., viral capsid, viral envelope, viral coat, viral accessory, viral glycoproteins, viral spike, etc.), extracellular and intracellular proteins, antibodies, and antigen binding fragments. In some cases, the analyte(s) can be localized to subcellular location(s), including, for example, organelles, e.g., mitochondria, Golgi apparatus, endoplasmic reticulum, chloroplasts, endocytic vesicles, exocytic vesicles, vacuoles, lysosomes, etc. In some cases, analyte(s) can be peptides or proteins, including without limitation antibodies and enzymes. Additional examples of analytes can be found in Section (I)(c) of WO 2020/176788 and/or U.S. Patent Application Publication No. 2020/0277663. In some cases, an analyte can be detected indirectly, such as through detection of an intermediate agent, for example, a ligation product or an analyte capture agent (e.g., an oligonucleotide-conjugated antibody), such as those described herein.

A “biological sample” is typically obtained from the subject for analysis using any of a variety of techniques including, but not limited to, biopsy, surgery, and laser capture microscopy (LCM), and generally includes cells and/or other biological material from the subject. In some cases, a biological sample can be a tissue section. In some cases, a biological sample can be a fixed and/or stained biological sample (e.g., a fixed and/or stained tissue section). Non-limiting examples of stains include histological stains (e.g., hematoxylin and/or eosin) and immunological stains (e.g., fluorescent stains). In some cases, a biological sample (e.g., a fixed and/or stained biological sample) can be imaged. Biological samples are also described in Section (I)(d) of WO 2020/176788 and/or U.S. Patent Application Publication No. 2020/0277663.

In some cases, a biological sample is permeabilized with one or more permeabilization reagents. For example, permeabilization of a biological sample can facilitate analyte capture. Exemplary permeabilization agents and conditions are described in Section (I)(d)(ii)(13) or the Exemplary Embodiments Section of WO 2020/176788 and/or U.S. Patent Application Publication No. 2020/0277663.

Array-based spatial analysis methods involve the transfer of one or more analytes from a biological sample to an array of features on a substrate, where each feature is associated with a unique spatial location on the array. Subsequent analysis of the transferred analytes includes determining the identity of the analytes and the spatial location of the analytes within the biological sample. The spatial location of an analyte within the biological sample is determined based on the feature to which the analyte is bound (e.g., directly or indirectly) on the array, and the feature's relative spatial location within the array.

A “capture probe” refers to any molecule capable of capturing (directly or indirectly) and/or labelling an analyte (e.g., an analyte of interest) in a biological sample. In some cases, the capture probe is a nucleic acid or a polypeptide. In some cases, the capture probe includes a barcode (e.g., a spatial barcode and/or a unique molecular identifier (UMI) sequence) and a capture domain). It is preferred that a molecular identifier be unique, that is generated randomly with minimal chance of repeating one sequence a second time. By having unique molecular identifiers, each captured analyte can be separately identifiable and therefore tracked via sequencing data for downstream analysis. In some cases, a capture probe can include a cleavage domain and/or a functional domain (e.g., a primer-binding site, such as for next-generation sequencing (NGS)). See, e.g., Section (II)(b) (e.g., subsections (i)-(vi)) of WO 2020/176788 and/or U.S. Patent Application Publication No. 2020/0277663. Generation of capture probes can be achieved by any appropriate method, including those described in Section (II)(d)(ii) of WO 2020/176788 and/or U.S. Patent Application Publication No. 2020/0277663.

In some cases, more than one analyte type (e.g., nucleic acids and proteins) from a biological sample can be detected (e.g., simultaneously or sequentially) using any appropriate multiplexing technique, such as those described in Section (IV) of WO 2020/176788 and/or U.S. Patent Application Publication No. 2020/0277663.

There are at least two methods to associate a spatial barcode with one or more neighboring cells, such that the spatial barcode identifies the one or more cells, and/or contents of the one or more cells, as associated with a particular spatial location. One method is to promote analytes or analyte proxies (e.g., intermediate agents) out of a cell and towards a spatially-barcoded array (e.g., including spatially-barcoded capture probes). Another method is to cleave spatially-barcoded capture probes from an array and promote the spatially-barcoded capture probes towards and/or into or onto the biological sample.

In some cases, capture probes may be configured to prime, replicate, and consequently yield optionally barcoded extension products from a template (e.g., a DNA or RNA template, such as an analyte or an intermediate agent (e.g., a ligation product or an analyte capture agent), or a portion thereof), or derivatives thereof (see, e.g., Section (II)(b)(vii) of WO 2020/176788 and/or U.S. Patent Application Publication No. 2020/0277663 regarding extended capture probes). In some cases, capture probes may be configured to form ligation products with a template (e.g., a DNA or RNA template, such as an analyte or an intermediate agent, or portion thereof), thereby creating ligations products that serve as proxies for a template.

As used herein, an “extended capture probe” refers to a capture probe having additional nucleotides added to the terminus (e.g., 3′ or 5′ end) of the capture probe thereby extending the overall length of the capture probe. For example, an “extended 3′ end” indicates additional nucleotides were added to the most 3′ nucleotide of the capture probe to extend the length of the capture probe, for example, by polymerization reactions used to extend nucleic acid molecules including templated polymerization catalyzed by a polymerase (e.g., a DNA polymerase or a reverse transcriptase). In some cases, extending the capture probe includes adding to a 3′ end of a capture probe a nucleic acid sequence that is complementary to a nucleic acid sequence of an analyte or intermediate agent specifically bound to the capture domain of the capture probe. In some cases, the capture probe is extended using reverse transcription. In some cases, the capture probe is extended using one or more DNA polymerases. The extended capture probes include the sequence of the capture probe and the sequence of the spatial barcode of the capture probe.

In some cases, extended capture probes are amplified (e.g., in bulk solution or on the array) to yield quantities that are sufficient for downstream analysis, e.g., via DNA sequencing. In some cases, extended capture probes (e.g., DNA molecules) act as templates for an amplification reaction (e.g., a polymerase chain reaction).

Additional variants of spatial analysis methods, including in some cases, an imaging step, are described in Section (II)(a) of WO 2020/176788 and/or U.S. Patent Application Publication No. 2020/0277663. Analysis of captured analytes (and/or intermediate agents or portions thereof), for example, including sample removal, extension of capture probes, sequencing (e.g., of a cleaved extended capture probe and/or a cDNA molecule complementary to an extended capture probe), sequencing on the array (e.g., using, for example, in situ hybridization or in situ ligation approaches), temporal analysis, and/or proximity capture, is described in Section (II)(g) of WO 2020/176788 and/or U.S. Patent Application Publication No. 2020/0277663. Some quality control measures are described in Section (II)(h) of WO 2020/176788 and/or U.S. Patent Application Publication No. 2020/0277663.

Spatial information can provide information of biological and/or medical importance. For example, the methods and compositions described herein can allow for: identification of one or more biomarkers (e.g., diagnostic, prognostic, and/or for determination of efficacy of a treatment) of a disease or disorder; identification of a candidate drug target for treatment of a disease or disorder; identification (e.g., diagnosis) of a subject as having a disease or disorder; identification of stage and/or prognosis of a disease or disorder in a subject; identification of a subject as having an increased likelihood of developing a disease or disorder; monitoring of progression of a disease or disorder in a subject; determination of efficacy of a treatment of a disease or disorder in a subject; identification of a patient subpopulation for which a treatment is effective for a disease or disorder; modification of a treatment of a subject with a disease or disorder; selection of a subject for participation in a clinical trial; and/or selection of a treatment for a subject with a disease or disorder. Exemplary methods for identifying spatial information of biological and/or medical importance can be found in U.S. Patent Application Publication No. 2021/0140982A1, U.S. Patent Application No. 2021/0198741A1, and/or U.S. Patent Application No. 2021/0199660.

Spatial information can provide information of biological importance. For example, the methods and compositions described herein can allow for: identification of transcriptome and/or proteome expression profiles (e.g., in healthy and/or diseased tissue); identification of multiple analyte types in close proximity (e.g., nearest neighbor analysis); determination of up- and/or down-regulated genes and/or proteins in diseased tissue; characterization of tumor microenvironments; characterization of tumor immune responses; characterization of cells types and their co-localization in tissue; and identification of genetic variants within tissues (e.g., based on gene and/or protein expression profiles associated with specific disease or disorder biomarkers).

Typically, for spatial array-based methods, a substrate functions as a support for direct or indirect attachment of capture probes to features of the array. A “feature” is an entity that acts as a support or repository for various molecular entities used in spatial analysis. In some cases, some or all of the features in an array are functionalized for analyte capture. Exemplary substrates are described in Section (II)(c) of WO 2020/176788 and/or U.S. Patent Application Publication No. 2020/0277663. Exemplary features and geometric attributes of an array can be found in Sections (II)(d)(i), (II)(d)(iii), and (II)(d)(iv) of WO 2020/176788 and/or U.S. Patent Application Publication No. 2020/0277663.

Generally, analytes and/or intermediate agents (or portions thereof) can be captured when contacting a biological sample with a substrate including capture probes (e.g., a substrate with capture probes embedded, spotted, printed, fabricated on the substrate, or a substrate with features (e.g., beads, wells) comprising capture probes). As used herein, “contact,” “contacted,” and/or “contacting,” a biological sample with a substrate refers to any contact (e.g., direct or indirect) such that capture probes can interact (e.g., bind covalently or non-covalently (e.g., hybridize)) with analytes from the biological sample. Capture can be achieved actively (e.g., using electrophoresis) or passively (e.g., using diffusion). Analyte capture is further described in Section (II)(e) of WO 2020/176788 and/or U.S. Patent Application Publication No. 2020/0277663.

In some cases, spatial analysis can be performed by attaching and/or introducing a molecule (e.g., a peptide, a lipid, or a nucleic acid molecule) having a barcode (e.g., a spatial barcode) to a biological sample (e.g., to a cell in a biological sample). In some cases, a plurality of molecules (e.g., a plurality of nucleic acid molecules) having a plurality of barcodes (e.g., a plurality of spatial barcodes) are introduced to a biological sample (e.g., to a plurality of cells in a biological sample) for use in spatial analysis. In some cases, after attaching and/or introducing a molecule having a barcode to a biological sample, the biological sample can be physically separated (e.g., dissociated) into single cells or cell groups for analysis. Some such methods of spatial analysis are described in Section (III) of WO 2020/176788 and/or U.S. Patent Application Publication No. 2020/0277663.

During analysis of spatial information, sequence information for a spatial barcode associated with an analyte is obtained, and the sequence information can be used to provide information about the spatial distribution of the analyte in the biological sample. Various methods can be used to obtain the spatial information. In some cases, specific capture probes and the analytes they capture are associated with specific locations in an array of features on a substrate. For example, specific spatial barcodes can be associated with specific array locations prior to array fabrication, and the sequences of the spatial barcodes can be stored (e.g., in a database) along with specific array location information, so that each spatial barcode uniquely maps to a particular array location.

Alternatively, specific spatial barcodes can be deposited at predetermined locations in an array of features during fabrication such that at each location, only one type of spatial barcode is present so that spatial barcodes are uniquely associated with a single feature of the array. Where necessary, the arrays can be decoded using any of the methods described herein so that spatial barcodes are uniquely associated with array feature locations, and this mapping can be stored as described above.

When sequence information is obtained for capture probes and/or analytes during analysis of spatial information, the locations of the capture probes and/or analytes can be determined by referring to the stored information that uniquely associates each spatial barcode with an array feature location. In this manner, specific capture probes and captured analytes are associated with specific locations in the array of features. Each array feature location represents a position relative to a coordinate reference point (e.g., an array location, a fiducial marker) for the array. Accordingly, each feature location has an “address” or location in the coordinate space of the array.

Some exemplary spatial analysis workflows are described in the Exemplary Embodiments section of WO 2020/176788 and/or U.S. Patent Application Publication No. 2020/0277663. See, for example, the Exemplary embodiment starting with “In some non-limiting examples of the workflows described herein, the sample can be immersed . . . ” of WO 2020/176788 and/or U.S. Patent Application Publication No. 2020/0277663. See also, e.g., the Visium Spatial Gene Expression Reagent Kits User Guide (e.g., Rev C, dated June 2020), and/or the Visium Spatial Tissue Optimization for FFPE Gene Expression Reagent Kits User Guide (e.g., Rev C, dated July 220 November 2021).

In some cases, spatial analysis can be performed using dedicated hardware and/or software, such as any of the systems described in Sections (II)(e)(ii) and/or (V) of WO 2020/176788 and/or U.S. Patent Application Publication No. 2020/0277663, or any of one or more of the devices or methods described in Sections Control Slide for Imaging, Methods of Using Control Slides and Substrates for, Systems of Using Control Slides and Substrates for Imaging, and/or Sample and Array Alignment Devices and Methods, Informational Labels of WO 2020/123320.

Suitable systems for performing spatial analysis can include components such as a chamber (e.g., a flow cell or sealable, fluid-tight chamber) for containing a biological sample. The biological sample can be mounted for example, in a biological sample holder. One or more fluid chambers can be connected to the chamber and/or the sample holder via fluid conduits, and fluids can be delivered into the chamber and/or sample holder via fluidic pumps, vacuum sources, or other devices coupled to the fluid conduits that create a pressure gradient to drive fluid flow. One or more valves can also be connected to fluid conduits to regulate the flow of reagents from reservoirs to the chamber and/or sample holder.

The systems can optionally include a control unit that includes one or more electronic processors, an input interface, an output interface (such as a display), and a storage unit (e.g., a solid state storage medium such as, but not limited to, a magnetic, optical, or other solid state, persistent, writeable and/or re-writeable storage medium). The control unit can optionally be connected to one or more remote devices via a network. The control unit (and components thereof) can generally perform any of the steps and functions described herein. Where the system is connected to a remote device, the remote device (or devices) can perform any of the steps or features described herein. The systems can optionally include one or more detectors (e.g., CCD, CMOS) used to capture images. The systems can also optionally include one or more light sources (e.g., LED-based, diode-based, lasers) for illuminating a sample, a substrate with features, analytes from a biological sample captured on a substrate, and various control and calibration media.

The systems can optionally include software instructions encoded and/or implemented in one or more of tangible storage media and hardware components such as application specific integrated circuits. The software instructions, when executed by a control unit (and in particular, an electronic processor) or an integrated circuit, can cause the control unit, integrated circuit, or other component executing the software instructions to perform any of the method steps or functions described herein.

In some cases, the systems described herein can detect (e.g., register an image) the biological sample on the array. Exemplary methods to detect the biological sample on an array are described in WO 2021/102003 and/or U.S. patent application Ser. No. 16/951,854, each of which is incorporated herein by reference in their entireties.

Prior to transferring analytes from the biological sample to the array of features on the substrate, the biological sample can be aligned with the array. Alignment of a biological sample and an array of features including capture probes can facilitate spatial analysis, which can be used to detect differences in analyte presence and/or level within different positions in the biological sample, for example, to generate a three-dimensional map of the analyte presence and/or level. Exemplary methods to generate a two- and/or three-dimensional map of the analyte presence and/or level are described in PCT Application No. 2020/053655 and spatial analysis methods are generally described in WO 2021/102039 and/or U.S. patent application Ser. No. 16/951,864, each of which is incorporated herein by reference in their entireties.

In some cases, a map of analyte presence and/or level can be aligned to an image of a biological sample using one or more fiducial markers, e.g., objects placed in the field of view of an imaging system which appear in the image produced, as described in the Substrate Attributes Section, Control Slide for Imaging Section of WO 2020/123320, WO 2021/102005, and/or U.S. patent application Ser. No. 16/951,843, each of which is incorporated herein by reference in their entireties. Fiducial markers can be used as a point of reference or measurement scale for alignment (e.g., to align a sample and an array, to align two substrates, to determine a location of a sample or array on a substrate relative to a fiducial marker) and/or for quantitative measurements of sizes and/or distances.

### Modified or Split Mirrored Transposon (SMT) Compositions

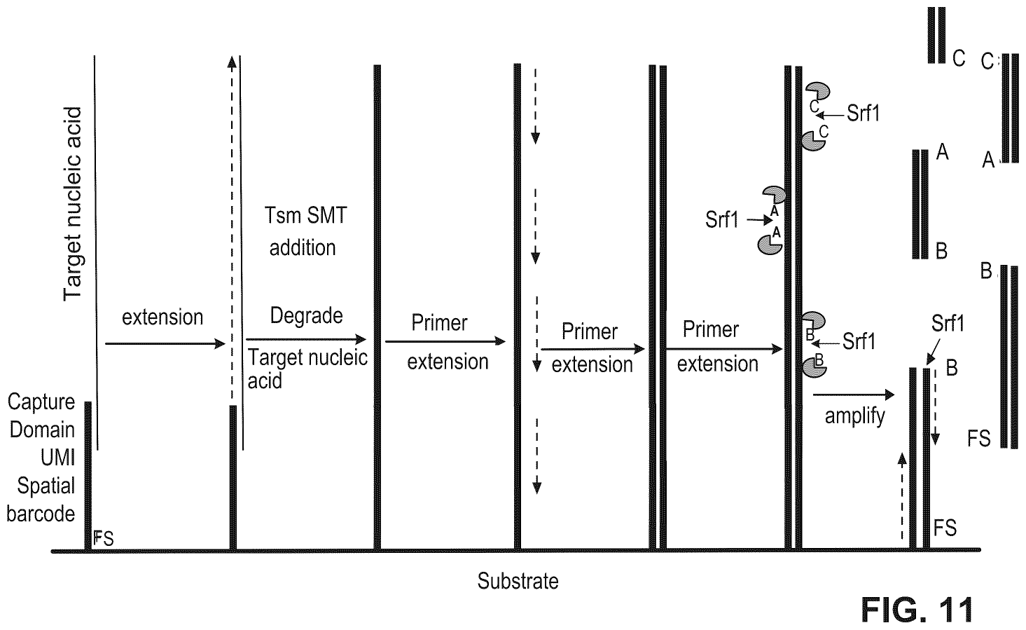

SMTs can also be in spatial analysis workflows to provide spatial information of nucleic acids from a biological sample (see FIG. 11). The use of SMTs can be used alone, for spatial analysis of a biological sample, or in combination with additional spatial analysis methods as described above. Nucleic acids such as genomic DNA or RNA sequences contain extensive biological information. Some sequencing technologies are only able to sequence short (e.g. <1000 bp) nucleic acid sequences, for example, because of library preparation limitations or sequencing technology limitations. Split mirrored transposon (SMT) compositions can provide long read sequence information by incorporating a unique sequence, such as a molecular identifier sequence, into a transposon containing sequence to identify upon sequencing nucleic acid ends that would have been connected in the cell or tissue, thereby preserving contiguity of a nucleic acid from a biological sample.

Generally, transposition is the process by which a specific genetic sequence (e.g., a transposon sequence) is relocated from one place in a genome to another. Many transposition methods and transposable elements are known in the art (e.g., DNA transposons, retrotransposons, autonomous transposons, non-autonomous transposons). One non-limiting example of a transposition event is conservative transposition. Conservative transposition is a non-replicative mode of transposition in which the transposon is completely removed from the genome and reintegrated into a new locus, such that the transposon sequence is conserved, (e.g., a conservative transposition event can be thought of as a “cut and paste” event). (See, e.g., Griffiths A. J., et. al., Mechanism of transposition in prokaryotes. An Introduction to Genetic Analysis (7th Ed.). New York: W. H. Freeman (2000)).

In one example, cut and paste transposition can occur when a transposase enzyme binds a sequence flanking the ends of the transposon (e.g., a recognition sequence, e.g., a mosaic end sequence). A transposome (a transposase complexed with transposon sequences) forms and the endogenous DNA can be manipulated into a pre-excision complex such that two transposase enzymes can interact. In some cases, when the transposases interact double stranded breaks are introduced into the DNA resulting in the excision of the transposon sequence. The transposase enzymes can locate and bind a target site in the DNA, create a double stranded break, and insert the transposon end sequence (See, e.g., Skipper, K. A., et. al., DNA transposon-based gene vehicles-scenes from an evolutionary drive, J Biomed Sci., 20: 92 (2013) doi:10.1186/1423-0127-20-92). Alternative cut and paste transposases include Tn552 (College, et al, J. BacterioL, 183: 2384-8, 2001; Kirby C et al, Mol. Microbiol, 43: 173-86, 2002), Tyl (Devine & Boeke, Nucleic Acids Res., 22: 3765-72, 1994 and International Publication WO 95/23875), Transposon Tn7 (Craig, N L, Science. 271: 1512, 1996; Craig, N L, Review in: Curr Top Microbiol Immunol, 204:27-48, 1996), Tn/O and IS10 (Kleckner N, et al, Curr Top Microbiol Immunol, 204:49-82, 1996), Mariner transposase (Lampe D J, et al, EMBO J., 15: 5470-9, 1996), Tel (Plasterk R H, Curr. Topics Microbiol. Immunol, 204: 125-43, 1996), P Element (Gloor, G B, Methods Mol. Biol, 260: 97-114, 2004), Tn3 (Ichikawa & Ohtsubo, J Biol. Chem. 265: 18829-32, 1990), bacterial insertion sequences (Ohtsubo & Sekine, Curr. Top. Microbiol. Immunol. 204: 1-26, 1996), retroviruses (Brown, et al, Proc Natl Acad Sci USA, 86:2525-9, 1989), and retrotransposon of yeast (Boeke & Corces, Annu Rev Microbiol. 43:403-34, 1989). More examples include IS5, TnlO, Tn903, IS911, and engineered versions of transposase family enzymes. (See, for example, Zhang et al, (2009) PLoS Genet. 5:e1000689. Epub 2009 Oct. 16 or Wilson C. et al (2007) J. Microbiol. Methods 71:332-5).

Transposome-mediated fragmentation and tagging (“tagmentation”) is a process of transposase-mediated fragmentation and tagging of nucleic acid, often DNA. A transposome (also known as a transposome complex) is a complex of a transposase enzyme and DNA which comprises a transposon end sequence (also known as “transposase recognition sequence” or “mosaic end” (MEs)). In some cases, DNA is fragmented in such a manner that a functional sequence such as a sequence complementary to a capture domain of a capture probe (e.g., capture domain of a splint oligonucleotide) is inserted into the fragmented DNA (e.g., the fragmented DNA is “tagged”), such that the sequence (e.g. an adapter) can hybridize to the capture probe. In some cases, the capture probe is present on a substrate. In some cases, the capture probe (e.g., a capture probe and a splint oligonucleotide) is present on a feature. In some cases, a transposase dimerizes to for a transposase dimer before interacting with a nucleic acid. A transposase dimer can then bind the nucleic acid with one of the transposases then recruit a second. In some cases, once a transposome complex is formed, a transposome complex dimer is formed. A transposome dimer (or more than two, referred to as a transposome multimer) is able to simultaneously fragment DNA based on its transposon recognition sequences and ligate DNA from the transposome to the fragmented DNA (e.g., tagmented DNA). See, for example, Blundell-Hunter et al., Nucleic Acids Research, 2018, 46:18, 9637-9646. This system has been adapted using hyperactive transposase enzymes and modified DNA molecules (adaptors) comprising MEs to fragment DNA and tag both strands of DNA duplex fragments with functional DNA molecules (e.g., primer binding sites). For instance, the Tn5 transposase may be produced as purified protein monomers. Tn5 transposase is also commercially available (e.g., Illumina, Illumina.com, Catalog No. 15027865, TD Tagment DNA Buffer Catalog No. 15027866). These can be subsequently loaded with the oligonucleotides of interest, e.g., ssDNA oligonucleotides containing MEs (e.g., transposon sequences) for Tn5 recognition and additional functional sequences (e.g., Nextera adapters, e.g., primer binding sites) are annealed to form a dsDNA mosaic end oligonucleotide (MEDS) that is recognized by Tn5 during dimer assembly (e.g., transposome dimerization). In some cases, a hyperactive Tn5 transposase can be loaded with adapters (e.g., oligonucleotides of interest) which can simultaneously fragment and tag a genome with the sequences.

The SMT compositions discloses herein include multiple different sequences setting it apart from traditional transpositional systems, in that contiguity is preserved and can be determined for a nucleic acid from a biological sample. For instance, in some cases, the SMT composition includes a first restriction enzyme site and a second restriction enzyme site. The first restriction enzyme site and a second restriction enzyme site (i.e., either the first restriction enzyme site or the second restriction enzyme site) can include a sequence recognized by one or more restriction enzymes or endonucleases. In some cases, any of the restriction enzyme site sequences are recognized by an endonuclease, such as a nicking endonuclease. Nicking endonucleases cut one strand of a double-stranded nucleic acid at a specific sequence rather than cutting both strands of the double-stranded nucleic acid. In some instances, nicking endonucleases recognize restriction enzyme site sequences that are 6 bp, 7 bp, or 8 bp long. Non-limiting exemplary nicking endonucleases include Nt.BstNBI, Nb.BsrDI, Nb.BtsI, Nt.AlwI, Nb.BbvCi, Nt.BbvCII, N.BspQI, Nt.BsmAI, Nt.BstNBI, Nt.CviPII, Nb.BssSI, and Nb.BsmI (see, for example. Walker, G. T. et al. (1992) Proc. Natl. Acad. Sci. USA, 89, 392-396; Wang, H. and Hays, J. B. (2000) Mol. Biotechnol., 15, 97-104; Higgins, L. S. et al. (2001) Nucleic Acids Res., 29, 2492-2501; Morgan, R. D. et al. (2000) Biol. Chem., 381, 1123-1125; Xu, Y. et al. (2001) Proc. Natl. Acad. Sci. USA, 98, 12990-12995; Heiter, D. F. et al. (2005) J. Mol. Biol., 348, 631-40; Samuelson, J. C., Zhu, Z. and Xu, S. Y. (2004) Nucleic Acids Res., 32, 3661-3671; Zhu, Z. et al. (2004) J. Mol. Biol., 337, 573-583). In some instances, the first and second restriction enzyme site sequences are different. In some instances, the first restriction enzyme site sequence is the NTt.BspQI restriction enzyme site sequence or the Nb.BsrDI restriction enzyme site sequence. In some instances, the second restriction enzyme site sequence is the NTt.BspQI restriction enzyme site sequence or the Nb.BsrDI restriction enzyme site sequence. In some instances, the first restriction enzyme site sequence is the Nt.BspQI restriction enzyme site sequence. In some instances, the second restriction enzyme site sequence is the Nb.BsrDI restriction enzyme site sequence.

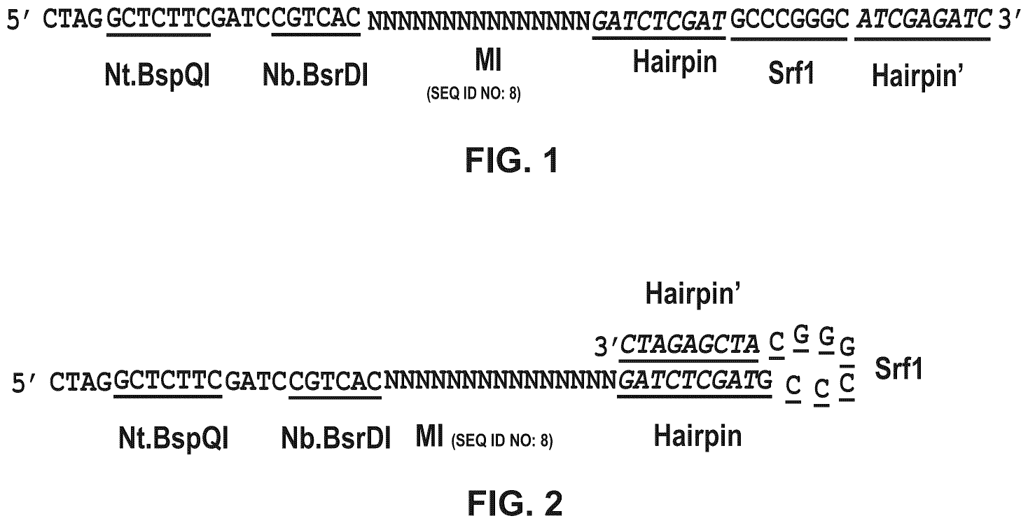

In some instances, a first and a second restriction enzyme site are adjacently positioned at the 5′ end of an oligonucleotide. A first restriction enzyme site and a second restriction enzyme site can be separated by at least 1 nucleotide to at least 100 nucleotides (e.g. at least 1 nucleotide to at least 10 nucleotides, 3 to 13, 5 to 15, 10 to 20, 20 to 30, 30 to 40, 40 to 50, 50 to 75, or 75 to 100 nucleotides). In some cases, preceding the first restriction enzyme site at the 5′ end is at least one to at least ten nucleotides. In some cases, the second restriction enzyme site sequence has a molecular identifier sequence (MI) 3′ of the second restriction enzyme site sequence (for example, see FIG. 1).

In some instances, the SMT compositions comprise a third restriction enzyme site sequence. The third restriction enzyme site sequence can include a sequence recognized by a restriction enzyme or an endonuclease. In some cases, the third restriction enzyme site sequence is recognized by a restriction enzyme, for example a Type I, Type II, or Type III restriction enzyme. Type II restriction enzymes cut at specific positions closer to or within the restriction enzyme sites thereby producing discrete restriction fragments. Restriction enzymes generate two different types of cuts; blunt ends are produced when the restriction enzyme cuts both strands of the nucleic acid at the same nucleotide in the restriction enzyme site, and sticky ends are produced when the restriction enzyme cuts each strand of the nucleic acid at a different nucleotide in the restriction enzyme site. In some cases, the third restriction enzyme site sequence is a 6 bp sequence, a 7 bp sequence, or a 8 bp sequence. In some cases, the third restriction enzyme site is unique to the genome (a unique restriction enzyme site), meaning that the restriction enzyme site is rare (e.g. a rare restriction enzyme site occurs less than 1000, 100, or 10 times in a genome). In some cases, the third restriction enzyme site is a NotI restriction enzyme site or a Srf1 restriction enzyme site. In some cases, the third restriction enzyme site sequence is recognized by a Srf1 restriction enzyme.

In some instances, the SMT compositions comprise a molecular identifier (MI) sequence. A MI is a contiguous nucleic acid segment of two or more non-contiguous nucleic acid segments that function as a label or identifier of a particular nucleic acid. A MI can be unique to a SMT. A MI can include one or more specific polynucleotides sequences, one or more random nucleic acid sequences, and/or one or more synthetic nucleic acid sequences, or combinations thereof.

In some cases, the MI is a nucleic acid sequence that does not substantially hybridize to native nucleic acid molecules found in a biological sample. In some cases, the MI has less than 80% sequence identity (e.g., less than 70%, 60%, 50%, or less than 40% sequence identity) to a substantial part (e.g., 80% or more) of the native nucleic acid molecules in the biological sample.

The MI can include from about 6 to about 20 or more nucleotides within the sequence of the SMT. In some cases, the length of a MI sequence can be about 6, 7, 8, 9, 10, 11, 12, 13, 14, 15, 16, 17, 18, 19, 20 nucleotides or longer. In some cases, the length of a MI sequence can be at least about 6, 7, 8, 9, 10, 11, 12, 13, 14, 15, 16, 17, 18, 19, 20 nucleotides or longer.

In some cases, the length of a MI sequence is at most about 6, 7, 8, 9, 10, 11, 12, 13, 14, 15, 16, 17, 18, 19, 20 nucleotides or shorter. In preferred embodiments, a MI is a random sequence specific to each SMT.

The nucleotides of MIs can be contiguous, i.e., in a single stretch of adjacent nucleotides, or they can be separated into two or more separate subsequences that are separated by 1 or more nucleotides (e.g., by 10, 15, 20, 25, 30, 35, 40, 45 or more nucleotides, or longer). Separated MI subsequences can be from about 4 to about 16 nucleotides in length. In some cases, the MI subsequence can be about 4, 5, 6, 7, 8, 9, 10, 11, 12, 13, 14, 15, 16 nucleotides or longer. In some cases, the MI subsequence can be at least about 4, 5, 6, 7, 8, 9, 10, 11, 12, 13, 14, 15, 16 nucleotides or longer. In some cases, the MI subsequence can be at most about 4, 5, 6, 7, 8, 9, 10, 11, 12, 13, 14, 15, 16 nucleotides or shorter. In some cases, MI subsequences can be separated by at least two hairpin sequences and a third restriction enzyme site sequence.

In some instances, the SMT compositions comprise a hairpin structure. The hairpin structure, or simply hairpin, comprises a double-stranded section referred to as a stem in which the DNA or RNA is self-complimentary and a single stranded section, referred to as a loop, that connects the ends of the double-stranded section on the same side of the molecule. The stem can be at least 5, 10, 15, 20, 25, 30, 35, 40, or 45 nucleotides long (e.g. 20, 21, 22, 23, 24, 25, 26, 27, 28, 29, or 30 nucleotides long). The loop can be at least 5, 10, 15, or 20 nucleotides long. In some case, the loop encodes the third restriction enzyme site sequence. In some cases, the hairpin is cut with a restriction enzyme that recognizes the restriction enzyme site sequence in the loop.

The SMT compositions further comprise transposon end sequences, also referred to as “mosaic ends”. The mosaic end or transposon sequence is specific to its transposase and inserts into a nucleic acid catalyzed by a transposase enzyme, the transposon sequences complexed with a transposase are collectively called a “transposome”. Mosaic ends are attached to the 5′ and 3′ end of the oligonucleotide either through chemical synthesis or through primer binding and extension with, for example, using a polymerase.

A SMT to which a transposase is complexed can include, starting at one end of a double-stranded molecule, a mosaic end, a molecular identifier (MI) sequence, a hairpin sequence, a third restriction enzyme site, the reverse-compliment of the hairpin sequence, a second MI identical to the first MI, and the second mosaic end (FIG. 8). To this SMT, a transposase is complexed to yield a transposome that includes two identical MIs separated by a third restriction enzyme site (FIG. 9).

In some cases, the step of fragmenting the genomic DNA in cells of the biological sample comprises contacting the biological sample containing the genomic DNA with the transposase enzyme (e.g., a transposome, e.g., a reaction mixture (e.g., solution)) including a transposase), under any suitable conditions. In some cases, such suitable conditions result in the tagmentation of the genomic DNA (traditionally) of cells present in the biological sample. Typical conditions will depend on the transposase enzyme used and can be determined using routine methods known in the art. However, a transposome can also tagment any DNA, it does not have to be chromosomal DNA. For example, FIG. 11 demonstrates how transposition can be performed using the compositions disclosed herein on dsDNA molecules generated on a substrate for determining spatial location of nucleic acids from a biological sample. Suitable conditions can be conditions (e.g., buffer, salt, concentration, pH, temperature, time conditions) under which the transposase enzyme is functional, e.g., in which the transposase enzyme displays transposase activity, particularly tagmentation activity, in the biological sample wherein the tagmented products can be captured on a spatial array, on dsDNA that is generated from captured target nucleic acids from a biological sample on a spatial array, in a lysate comprising nucleic acids to be tagmented for example for sequence library preparation, or on a purified nucleic acid sample comprising nucleic acids to be tagmented for example for sequence library preparation.

The term “functional”, as used herein in reference to transposase enzymes, is meant to include embodiments in which the transposase enzyme can show some reduced activity relative to the activity of the transposase enzyme in conditions that are optimum for the enzyme, e.g., in the buffer, salt and temperature conditions recommended by the manufacturer. Thus, the transposase can be considered to be “functional” if it has at least about 50%, e.g., at least about 60%, about 70%, about 80%, about 85%, about 90%, about 95%, about 96%, about 97%, about 98%, about 99%, or about 100%, activity relative to the activity of the transposase in conditions that are optimum for the transposase enzyme.

In one non-limiting example, the reaction mixture comprises a transposome in a buffered solution (e.g., Tris-acetate) having a pH of about 6.5 to about 8.5, e.g., about 7.0 to about 8.0 such as about 7.5. Additionally or alternatively, the reaction mixture can be used at any suitable temperature, such as about 100 to about 55° C., e.g., about 10° to about 54°, about 11° to about 53°, about 120 to about 52°, about 130 to about 510, about 140 to about 50°, about 150 to about 49°, about 160 to about 48°, about 170 to about 47° C., e.g., about 10°, about 12°, about 15°, about 18°, about 20°, about 22°, about 25°, about 28°, about 30°, about 33°, about 35°, about or 37° C., preferably about 300 to about 40° C., e.g., about 37° C. In some cases, the transposome can be contacted with the biological sample for about 10 minutes to about one hour. In some cases, the transposome can be contacted with the biological sample for about 20, about 30, about 40, or about 50 minutes. In some cases, the transposome can be contacted with the biological sample for about 1 hour to about 4 hours.

In some cases, the transposase enzyme of the transposome complex is a Tn5 transposase, or a functional derivate or variant thereof. (See, e.g., Reznikoff et al, WO 2001/009363, U.S. Pat. Nos. 5,925,545, 5,965,443, 7,083,980, and 7,608,434, and Goryshin and Reznikoff, J. Biol. Chem. 273:7367, (1998), which are herein incorporated by reference). In some cases, the Tn5 transposase is a hyper Tn5 transposase, or a functional derivate or variate thereof (U.S. Pat. No. 9,790,476, incorporated herein by reference). For example, the Tn5 transposase can be a fusion protein (e.g., a Tn5 fusion protein). Tn5 is a member of the RNase superfamily of proteins. The Tn5 transposon is a composite transposon in which two near-identical insertion sequences (IS50L and IS50R) flank three antibiotic resistance genes. Each IS50 contains two inverted 19-bp end sequences (ESs), an outside end (OE) and an inside end (IE). Wild-type Tn5 transposase enzyme is generally inactive (e.g., low transposition event activity). However, amino acid substitutions can result in hyperactive variants or derivatives. In one non-limiting example, amino acid substitution, L372P, substitutes a leucine amino acid for a proline amino acid which results in an alpha helix break, thus inducing a conformational change to the C-terminal domain. The alpha helix break separates the C-terminal domain and N-terminal domain sufficiently to promote higher transposition event activity (See, Reznikoff, W. S., Tn5 as a model for understanding DNA transposition, Mol Microbiol, 47(5): 1199-1206 (2003)). Other amino acid substitutions resulting in hyperactive Tn5 are known in the art. For example, the improved avidity of the modified transposase enzyme (e.g., modified Tn5 transposase enzyme) for the repeat sequences for OE termini (class (1) mutation) can be achieved by providing a lysine residue at amino acid 54, which is glutamic acid in wild-type Tn5 transposase enzyme (See U.S. Pat. No. 5,925,545). The mutation strongly alters the preference of the modified transposase enzyme (e.g., modified Tn5 transposase enzyme) for OE termini, as opposed to IE termini. The higher binding of this mutation, known as EK54, to OE termini results in a transposition rate that is about 10-fold higher than is seen with wild-type transposase enzyme (e.g., wild type Tn5 transposase enzyme). A similar change at position 54 to valine (e.g., EV54) also results in somewhat increased binding/transposition for OE termini, as does a threonine to proline change at position 47 (e.g., TP47; about 10-fold higher). (See, for example, U.S. Pat. No. 5,925,545.)

Other examples of modified transposase enzymes (e.g., modified Tn5 transposase enzymes) are known. For example, a modified Tn5 transposase enzyme that differs from wild-type Tn5 transposase enzyme in that it binds to the repeat sequences of the donor DNA with greater avidity than wild-type Tn5 transposase enzyme and also is less likely than the wild-type transposase enzyme to assume an inactive multimeric form (U.S. Pat. No. 5,925,545, which is incorporated by reference in its entirety). Furthermore, techniques generally describing introducing any transposable element (e.g., Tn5) from a donor DNA (e.g., adapter sequence, e.g., Nextera adapters (e.g., top and bottom adapter) into a target are known in the art. (See, e.g., U.S. Pat. No. 5,925,545). Further study has identified classes of mutations resulting in a modified transposase enzyme (e.g., modified Tn5 transposase enzyme) (See, U.S. Pat. No. 5,965,443, which is incorporated by reference in its entirety). For example, a modified transposase enzyme (e.g., modified Tn5 transposase enzyme) with a “class 1 mutation” binds to repeat sequences of donor DNA with greater avidity than wild-type Tn5 transposase enzyme. Additionally, a modified transposase enzyme (e.g., modified Tn5 transposase enzyme) with a “class 2 mutation” is less likely than the wild-type Tn5 transposase enzyme to assume an inactive multimeric form. It has been shown that a modified transposase enzyme that contains both a class 1 and a class 2 mutation can induce at least about 100-fold (+10%) more transposition than the wild-type transposase enzyme, when tested in combination with an in vivo conjugation assay as described by Weinreich, M. D., “Evidence that the cis Preference of the Tn5 Transposase is Caused by Nonproductive Multimerization,” Genes and Development 8:2363-2374 (1994), incorporated herein by reference (See e.g., U.S. Pat. No. 5,965,443). Further, under sufficient conditions, transposition using the modified transposase enzyme (e.g., modified Tn5 transposase enzyme) may be higher. A modified transposase enzyme containing only a class 1 mutation can bind to the repeat sequences with sufficiently greater avidity than the wild-type Tn5 transposase enzyme such that a Tn5 transposase enzyme induces about 5- to about 50-fold more transposition than the wild-type transposase enzyme, when measured in vivo. A modified transposase enzyme containing only a class 2 mutation (e.g., a mutation that reduces the Tn5 transposase enzyme from assuming an inactive form) is sufficiently less likely than the wild-type Tn5 transposase enzyme to assume the multimeric form that such a Tn5 transposase enzyme also induces about 5- to about 50-fold more transposition than the wild-type transposase enzyme, when measured in vivo (See U.S. Pat. No. 5,965,443).

More embodiments of transposases and transposon nucleic acids useful with some of the methods and compositions provided herein include Staphylococcus aureus Tn552 (Colegio et al., J. Bacteriol., 183: 2384-8 (2001); Kirby et al., Mol. Microbiol., 43: 173-86 (2002)), Tyl (Devine & Boeke, Nucleic Acids Res., 22: 3765-72 (1994) and WO 95/23875), Transposon Tn7 (Craig, Science 271: 1512 (1996); Craig, Curr Top Microbiol Immunol., 204:27-48 (1996)), Tn/O and IS10 (Kleckner et al., Curr Top Microbiol Immunol., 204:49-82 (1996)), Mariner transposase (Lampe et al., EMBO J., 15: 5470-9, (1996)), Tel (Plasterk, Curro Topics Microbiol. Immunol., 204: 125-43, (1996)), P Element (Gloor, Methods Mol. Biol., 260: 97-114, (2004)), Mos-1 transposase (Richardson et al., EMBO Journal 25:1324-1334 (2006)), Tn3 (Ichikawa & Ohtsubo, J Biol. Chem. 265: 18829-32, (1990)), bacterial insertion sequences (Ohtsubo & Sekine, Curro Top. Microbiol. Immunol. 204: 1-26, (1996)), retroviruses (Brown, et al., Proc Natl Acad Sci USA, 86:2525-9, (1989)), and retrotransposon of yeast (Boeke & Corces, Annu Rev Microbiol. 43:403-34, (1989)). More examples include IS5, Tn10, Tn903, IS911, and engineered versions of transposase family enzymes (Zhang et al., PLoS Genet. 5:e1000689. Epub 2009 Oct. 16; and Wilson et al. Microbiol. Methods 71:332-5 (2007)). More examples include MuA transposases (See e.g., Rasila T S, et al., (2012) PLoS ONE 7(5): e37922. doi:10.1371/journal.pone.0037922) and Vibhar transposases (See, for example, U.S. Pat. No. 10,100,348). Each of the references cited in this paragraph is incorporated herein by reference in its entirety.

Other methods of using a modified transposase enzyme (e.g., modified Tn5 transposase enzyme) are further generally described in U.S. Pat. Nos. 5,965,443 and 9,790,476.

In some cases, the transposase enzyme, or functional variant or derivative thereof, comprises an amino acid sequence having at least 80% sequence identity to SEQ ID NOs. 1-5. In some cases, the Tn5 transposase enzyme, or functional variant or derivative thereof, comprises an amino acid sequence having a sequence identity of at least about 90, 91, 92, 93, 94, 95, 96, 97, 98, or 99% amino acid sequence identity to SEQ ID NOs. 1-5. In some cases, the transposase enzyme is a Tn5 transposase enzyme, or functional derivative thereof. In some cases, the Tn5 transposase enzyme, or functional variant or derivative thereof, comprises an amino acid sequence having at least 80% sequence identity to SEQ ID NO. 1. In some cases, the Tn5 transposase enzyme, or functional variant or derivative thereof, comprises an amino acid sequence having a sequence identity of at least about 90, 91, 92, 93, 94, 95, 96, 97, 98, or 99% amino acid sequence identity to SEQ ID NO. 1.

In some cases, the transposase is a Tn5 transposase enzyme, or a functional variant or derivative thereof and the transposon end sequences used in the SMT are recognized by and will complex with the Tn5 transposase enzyme. In some cases, the transposase enzyme is a Mu transposase enzyme, or a functional variant or derivative thereof and the transposon end sequences used in the SMT are recognized by and will complex with the Mu transposase. In some cases, the transposase is a Vibhar transposase, or functional variant or derivative thereof and the transposon end sequences used in the SMT are recognized by and will complex with a Vibhar transposase. In some cases, the transposase is a Mariner transposase, or functional variant or derivative thereof and the transposon end sequences used in the SMT are recognized by and will complex with a Mariner transposase. In some cases, the transposase is a Tn7 transposase, or functional variant or derivative thereof and the transposon end sequences used in the SMT are recognized by and will complex with a Tn7 transposase. The present disclosure is not limited to the type of transposase used, only that the transposon ends as appended to an oligonucleotide to generate a modified or split mirrored transposon are recognized by and will complex with the type of transposase.

A split mirrored transposon includes an oligonucleotide that comprises at either end a mosaic end transposon sequence, a first molecular identifier sequence, a hairpin sequence, a third restriction enzyme site sequence, a reverse-compliment of the hairpin sequence, a second molecular identifier sequence, and a second mosaic end, and a first and a second transposase, each attached to a mosaic end. In some cases, the first and second transposase are the same transposase. In some cases, the first and second transposases are functional variation or derivative of the same transposase. In some cases, the first transposase is a first Tn5 transposase or functional variation thereof, and the second transposase is a second Tn5 transposase or functional variation thereof, wherein the first Tn5 transposase and the second Tn5 transposase are different.

In certain cases, the oligonucleotide can be double-stranded DNA. In certain cases, the oligonucleotide can contain both single and double-stranded DNA components. In some cases, the single-stranded DNA component is attached to the double-stranded component, and the two strands of the double-stranded component are attached with a single-stranded loop, making a stem-and-loop structure attached to a single-stranded component. In some cases, the oligonucleotide contains a double-stranded component, and the two strands of the double-stranded component are attached with a single-stranded loop, making a stem-and-loop structure. In some cases, the single-stranded component is on the 5′ end of the oligonucleotide. In some cases, the single-stranded component is on the 3′ end of the oligonucleotide. In some case, there are multiple (e.g. two) single-stranded components. In some cases, the single-stranded components are made by nicking the oligonucleotide with a nicking endonuclease. Exemplary nicking endonucleases are described above. In some cases, single-stranded components can include mosaic ends to which transposases can attach. In some cases, double-stranded components can include mosaic ends to which transposases can attach.

### Methods of Generating Split Mirrored Transposons

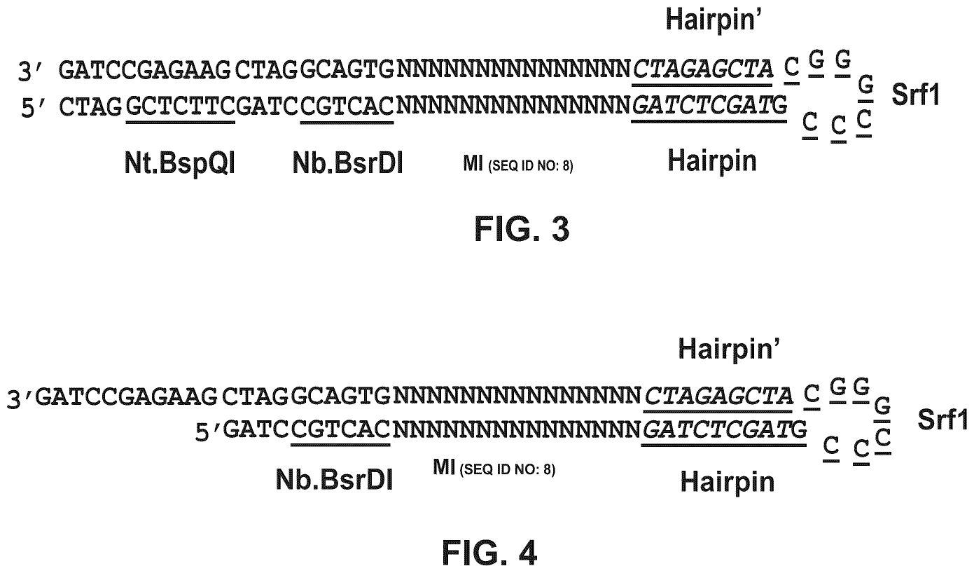

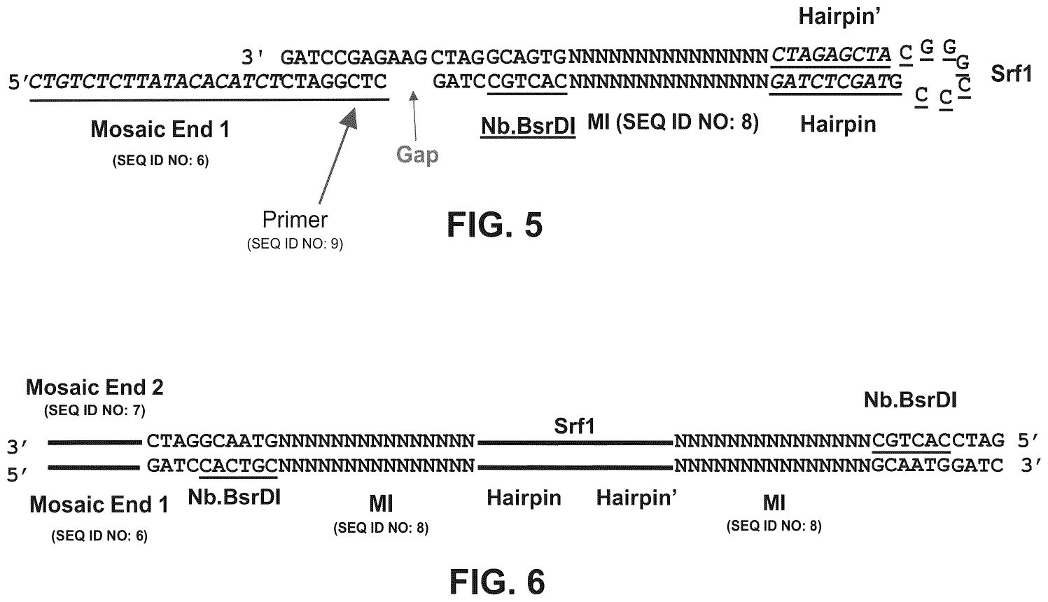

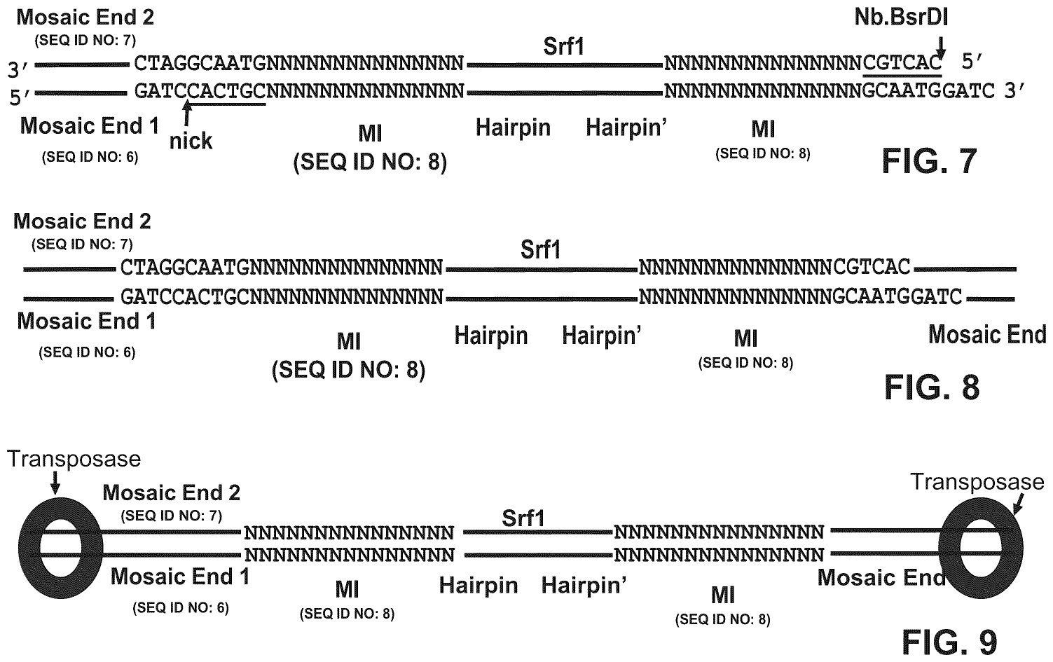

Provided herein are methods of generating or producing a split mirrored transposon, including a) providing an oligonucleotide sequence comprising: i) a first restriction enzyme site sequence, ii) a second restriction enzyme site sequence, iii) a molecular identifier sequence (MI), and iv) two hairpin sequences (a first hairpin sequence and a second hairpin sequence) that flank a third restriction enzyme site (FIG. 1), wherein the two hairpin sequences are substantially complementary to each other and hybridize to each other to create a hairpin loop, wherein the third restriction enzyme site is preferably unique in a target genome (FIG. 2), b) extending the hairpin loop to generate a double-stranded sequence comprising the MI and its complement, the first restriction enzyme site sequence and its complement, and the second restriction enzyme site and its complement (FIG. 3); c) digesting one strand of the double-stranded sequence with a nicking enzyme that recognizes the first restriction enzyme site sequence to generate a 3′ overhang at the site of the first restriction enzyme site sequence (FIG. 4); d) hybridizing a primer comprising a mosaic end at its 5′ end to the 3′ overhang (FIG. 5); e) generating a complete double-stranded nucleic acid molecule using a strand displacing enzyme thereby relieving the hairpin loop structure (FIG. 6); f) digesting the double-stranded nucleic acid molecule with a second restriction enzyme that generates a nick at one end of the molecule and generates a 3′ overhang on the other end (FIG. 7); g) ligating a duplexed mosaic end to the double-stranded sequence, thereby generating a double-stranded split mirrored nucleic acid (also called a double stranded transposon nucleic acid) comprising: i) two mosaic ends, ii) two MIs, and iii) a nucleic acid sequence comprising the two hairpin sequences separated by a third restriction enzyme site (FIG. 8); and h) adding one or more transposase enzymes that bind to mosaic ends to generate a double-stranded split mirrored transposome (also called a transposome complex) (FIG. 9).

In some cases, the nucleic acid molecule, SMT or oligonucleotide of the disclosure (and any intermediates thereof) is chemically synthesized. In some cases, the transposon is attached to a chemically synthesized oligonucleotide. In some cases, any of the intermediate oligonucleotides can be chemically synthesized. In some cases, generating or producing a split mirrored transposon can In some cases, annealing the primer comprising a mosaic end leaves a gap between the primer and the second restriction enzyme site sequence for a polymerase, such as a strand displacing polymerase to seal the gap (FIG. 5). In some cases, annealing the primer comprises a mosaic end that does not leave a gap between the primer and the second restriction enzyme site sequence. In some cases, the nucleotide backbone between the primer and the second restriction enzyme site sequence is sealed and then the hairpin is denatured with heat and the oligonucleotide is amplified using a primer complimentary to the 3′ end of the unfolded hairpin structure.

### Applications of Modified or Split Mirrored Transposon Compositions

There are numerous applications of split mirrored transposon compositions. For example, split mirrored transposons and compositions thereof can facilitate sequencing long nucleic acids in a variety of contexts to elucidate biological information. Further, the present methods can be used for inserting, for example, Cre or LoxP recombination sites which may be useful in genomic engineering methods. Also, the methods could be useful in inserting matching promoters, enhancers, or other regulatory elements like polycomb, HOX or hypoxia response elements (HRE) for downstream application is research and biotechnology development. Basically, the ability of the present methods to insert two identical and functional sequences adjacent to each other throughout genomic DNA can be useful in many research and investigatory efforts for cellular mechanisms, dysregulation in cancer research, etc.

### A) Providing SMTs to a Lysate

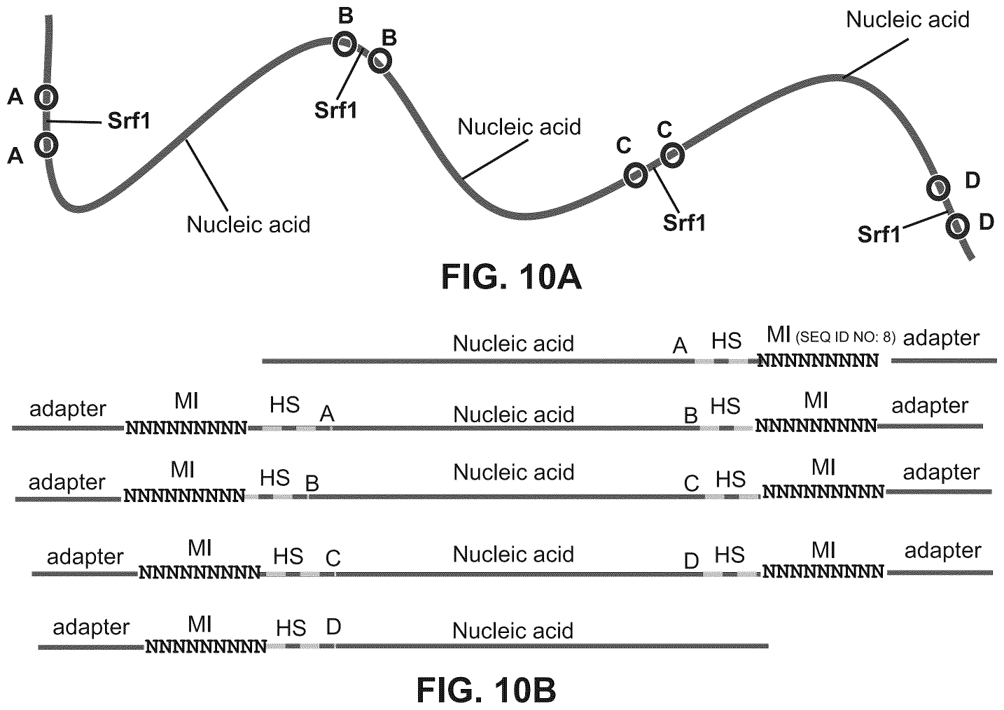

Disclosed herein are method of analyzing nucleic acids from a biological sample, such as a tissue, a cell or tissue lysate, or purified nucleic acids. In some cases, a lysate is produced from the biological sample. In some cases, nucleic acids is purified from the biological sample. In some cases, gDNA is purified from the biological sample. Methods in relation to a lysate or purified nucleic acids (e.g. purified gDNA) can include inserting the transposon into the nucleic acids of the lysate or into the purified gDNA; providing any of the compositions described herein and a transposase enzyme to the lysate of purified nucleic acids under conditions wherein the composition is inserted into the nucleic acids; allowing the transposase enzyme to excise the inserted transposon sequence from the nucleic acids of the lysate or the purified nucleic acids, thereby generating fragmented nucleic acids; and collecting the fragmented nucleic acids. For example, FIG. 10A shows how SMT complexes are added to a nucleic acid molecule from a lysate or a purified nucleic acid sample, such that tagmentation would yield the nucleic acid tagmented molecules as depicted in FIG. 10B. FIG. 10B further shows that adapters have been optionally added to ends of the tagmented products. Adapters can be used in sequencing workflows, additionally they can be used as capture domains, for example to capture the tagmented products on the surface of an array, bead, or other substrate by capture probes that are affixed to the surface of the substrate, wherein the capture probes could be spatially barcoded.

### B) Using SMTs for Sequencing Library Preparation

Disclosed herein are methods of preparing a library of nucleic acids from a biological sample, such as a tissue, a cell or tissue lysate, or purified nucleic acids. Methods in relation to a tissue sample can include permeabilizing the tissue sample under conditions sufficient to allow nucleic acids in the biological sample to be accessible to transposon insertion; providing any of the compositions described herein and a transposase enzyme to the tissue sample under conditions wherein the composition is inserted into the nucleic acids; allowing the transposase enzyme to excise the inserted transposon sequence from the nucleic acids, thereby generating fragmented nucleic acids; and collecting the fragmented nucleic acids.

In some cases, nucleic acids are pre-processed for library generation via next generation sequencing. For example, nucleic acids can be pre-processed by addition of a modification (e.g., ligation of sequences that allow interaction with capture probes). In some cases, nucleic acids (e.g., DNA or RNA) are fragmented using fragmentation techniques (e.g., using transposases and/or fragmentation buffers).

Fragmentation can be followed by a modification of the nucleic acid. For example, a modification can be the addition through ligation of an adapter sequence that allows hybridization with a capture probe on an array, for example for spatial determination of nucleic acids in a tissue sample. In some cases, where the analyte of interest is RNA, poly(A) tailing is performed. Addition of a poly(A) tail to RNA that does not contain a poly(A) tail can facilitate hybridization with a capture probe that includes a capture domain with a functional amount of poly(dT) sequence.

In some cases, prior to interaction with capture probes, ligation reactions catalyzed by a ligase are performed in the tissue sample. In some cases, ligation can be performed by chemical ligation. In some cases, the ligation can be performed using click chemistry as further described below. In some cases, the capture domain includes a DNA sequence that has complementarity to a RNA molecule, where the RNA molecule has complementarity to a second DNA sequence, and where the RNA-DNA sequence complementarity is used to ligate the second DNA sequence to the DNA sequence in the capture domain. In these cases, direct detection of RNA molecules is possible.

In some cases, prior to interaction with capture probes, target-specific reactions are performed in the tissue sample. Examples of target specific reactions include, but are not limited to, ligation of target specific adaptors, probes and/or other oligonucleotides, target specific amplification using primers specific to one or more nucleic acids, and target-specific detection using in situ hybridization, DNA microscopy, and/or antibody detection. In some cases, a capture probe includes capture domains targeted to target-specific products (e.g., amplification or ligation).

### C) Using SMTs for Single Cell Analysis

Disclosed herein are methods of analyzing nucleic acids present in a single cell biological sample. Such methods can include permeabilizing the single cell biological sample under conditions sufficient to allow the nucleic acid in the single cell biological sample to be accessible to transposon insertion; providing the compositions as disclosed herein and a transposase enzyme to the single cell biological sample under conditions wherein the composition is inserted into the nucleic acid; allowing the transposase enzyme to excise the inserted transposon sequence from the nucleic acid, thereby generating a fragmented nucleic acid; and analyzing the fragmented nucleic acid as an indicator of the presence of the nucleic acid in the single cell biological sample.

Also disclosed herein are methods for profiling at least one biological nucleic acid (e.g., DNA, RNA) present in a cell-containing biological sample. The methods disclosed herein include preparing a cell-containing biological sample such that it includes, in some cases, a suspension of single cells. The preparation of cells is added to a substrate as disclosed herein (e.g., that includes a plurality of probes comprising a spatial barcode and a capture domain). The preparation of cells is immobilized onto the substrate, thereby providing a distinct spatial location for single cells on the substrate.

Provided herein are methods of analyzing a biological sample that includes one or more single cells (e.g. a single cell or a plurality of single cells) and any of the split mirrored transposons or split mirrored transposon compositions described herein. In some cases, the biological sample is a cell-containing biological sample. As used herein, a “cell-containing biological sample” is a biological sample (e.g., a tissue sample, a liquid sample (e.g., blood, saliva, etc., a cell culture sample) that includes at least one cell. In some cases, a cell-containing biological sample includes more than one cell. In some cases, a cell-containing biological sample includes more than one cell type (e.g. a tissue section or tissue sample).

In some cases, the methods of analyzing include detecting the presence of one or more nucleic acids in a biological sample. In some cases, the biological sample is a single cell. In some cases, the biological sample is a collection of single cells (i.e., a plurality of cells). In some cases, a plurality of cells includes cells that are not aggregated to other cells, e.g., the plurality of cells is a plurality of single cells.

In some cases, a plurality of cells includes cells from a suspension of cells and/or dissociated cells from a tissue or tissue section. In some cases, a plurality of cells comprises cells from a disaggregated tissue or tissue section. In some cases, the plurality of cells includes cells from the same cell type. In some cases, the plurality of cells includes cells from a heterogeneous population of cells. For example, the plurality of cells can be from a tissue that has multiple cell types, such as a liver tissue containing hepatocytes, stellate cells, Kupffer cells, sinusoidal endothelial cells, cancerous liver cells, etc., or a kidney tissue containing glomerulus parietal cells, glomerulus podocytes, proximal tubule brush border cells, cancerous kidney cells, etc.

In some cases, the cells of a tissue or tissue section can be disassociated into disaggregated cells. For example, cells from a tissue or tissue section can be disassociated using any means known in the art. In some cases, cells from a tissue or tissue section are disassociated using enzymatic or mechanical means. Non-limiting examples of enzymes used in enzymatic disaggregation include dispase, collagenase, proteinase k, trypsin, or combinations thereof. In some cases, mechanical disaggregation includes a tissue homogenizer or dissociator.

In some cases, a plurality of cells comprises cells from a cell culture. In some cases, a cell culture includes adherent cells (e.g., cells that are anchorage-dependent). Non-limiting examples of adherent cells include DU145 (prostate cancer) cell, H295R (adrenocortical cancer) cell, HeLa (cervical cancer) cells, KBM-7 (chronic myelogenous leukemia) cells, LNCaP (prostate cancer) cells, MCF-7 (breast cancer) cells, MDA-MB-468 (breast cancer) cells, PC3 (prostate cancer) cells, SaOS-2 (bone cancer) cells, SH-SY5Y (neuroblastoma, cloned from a myeloma) cells, T-47D (breast cancer) cells, THP-1 (acute myeloid leukemia) cells, U87 (glioblastoma) cells, National Cancer Institute's 60 cancer cell line panel (NCI60), vero (African green monkey Chlorocebus kidney epithelial cell line) cells, MC3T3 (embryonic calvarium) cells, GH3 (pituitary tumor) cells, PC12 (pheochromocytoma) cells, dog MDCK kidney epithelial cells, Xenopus A6 kidney epithelial cells, zebrafish AB9 cells, and Sf9 insect epithelial cells. Additional examples of adherent cells are shown in Table 1. See, e.g., DTP, DCTD Tumor Repository. A Catalog of in Vitro Cell Lines, Transplantable Animal and Human Tumors and Yeast. The Division of Cancer Treatment and Diagnosis (DCTD), National Cancer Institute, 2013; and Abaan et al. The exomes of the NCI-60 panel: a genomic resource for cancer biology and systems pharmacology. Cancer Research. 2013; each of which are incorporated by reference herein in their entireties).

In some cases, a cell culture comprises suspension cells (e.g., cells that are anchorage-independent). Many adherent cell lines can also be cultured as a suspension of cells. Non-limiting examples of suspension cells include cell lines derived from hematopoietic cells. Other non-limiting examples of suspension cells include Colo205, CCRF-CEM, HL-60, K562, MOLT-4, RPMI-8226, SR, HOP-92, NCI-H322M, and MALME-3M. Methods for culturing cells such as from the cell lines described herein are well known to one of ordinary skill in the art.

In some cases, a plurality of cells can be obtained from non-mammalian organisms (e.g., a plant, an insect, an arachnid, a nematode (e.g., Caenorhabditis elegans), a fungi, an amphibian, or a fish (e.g., zebrafish)). Single cells can be obtained from a prokaryote such as a bacterium, e.g., Escherichia coli, Staphylococci or Mycoplasma pneumoniae; an archaea; a virus such as Hepatitis C virus or human immunodeficiency virus; or a viroid.

In some cases, nucleic acids from a cell are profiled. In some cases, a nucleic acid from a cell is profiled after the cell is immobilized onto a substrate as disclosed herein. In some cases, a probe affixed to the substrate hybridizes to the nucleic acid.

In some cases, the substrate includes a plurality of probes at known spatial locations. In this instance, cell doublets are captured. “Cell doublets” are artifactual libraries generated from two cells, sometimes seen in droplet-based sequencing when at least 2 cells are captured. See e.g., Zheng et al. Nat Commun. 2017 Jan. 16; 8:14049. Cell doublets occurring between distinct cell types can appear as hybrid scRNA-seq profiles, but do not have distinct transcriptomes from individual cell states. See DePasquale, Cell Rep. 2019 Nov. 5; 29(6):1718-1727.e8. In some cases, cell doublets are filtered and therefore excluded from downstream analysis. In some cases, additional downstream analysis includes pooling of barcodes.

In some cases, the nucleic acid is amplified using any of the amplification methods disclosed herein. In some cases, amplification occurs after the nucleic acid is released from the probe. In some cases, the nucleic acid is amplified. In some cases, only part of the nucleic acid is amplified. In some cases, amplification occurs before the nucleic acid is released from the probe. In some cases, amplification is isothermal. In some cases, amplification is not isothermal. Amplification can be performed using any of the methods described herein such as, but not limited to, a polymerase chain reaction (PCR) or an extension-ligation reaction as disclosed herein, a strand-displacement amplification reaction, a rolling circle amplification reaction, a ligase chain reaction, a transcription-mediated amplification reaction, an isothermal amplification reaction, and/or a loop-mediated amplification reaction. In some cases, amplifying the nucleic acid creates an amplified product that includes (i) all or part of the sequence of the nucleic acid specifically bound to the capture domain, or a complement thereof, and (ii) all or a part of the sequence of the spatial barcode, or a complement thereof.

In some cases, the amplified product is sequenced using any of the methods described herein. For example, in some cases, a library is constructed. In some cases, any of the next-generation sequencing methods described herein are used. In some cases, after sequencing, cell morphology is correlated with the sequencing information.

The methods described herein provide for the compartmentalization or partitioning of a cell from a biological sample into discrete partitions, or voxels. As used herein, each “voxel” represents a 3-dimensional volumetric unit. In some cases, a voxel maintains separation of its own contents from the contents of other voxels. A voxel can be one partition in a series of discrete partitions into which a three-dimensional object is divided. For example, a plurality of crosslinkable polymer precursors can be cross-linked into voxels that are part of a crosslinked polymer covering the substrate, or a portion of the substrate. Unique identifiers, e.g., barcodes, may be previously, subsequently or concurrently attached to the cell to allow for the later attribution of characteristics of the cell to the particular voxel. In some cases, a voxel has defined dimensions. In some cases, a voxel comprises a single cell. In some cases, a voxel is a single cell.

### D) Using SMTS for ATAC Sequencing Analysis

The human body includes a large collection of diverse cell types, each providing a specialized and context-specific function. Understanding a cell's chromatin structure (chromosomal DNA, genomic DNA) can reveal information about the cell's function. Open chromatin, or accessible chromatin that expression regulatory elements and transcription machinery can access or bind to, is often indicative of transcriptionally active sequences, e.g., genes, in a particular cell. Further understanding the transcriptionally active regions within chromatin will enable identification of which genes contribute to a cell's function and/or phenotype. Methods have been developed to study epigenomes, e.g., chromatin accessibility assays (Assay for Transposase Accessible Chromatin, or ATAC-seq) or identifying proteins associated with chromatin e.g., (Chromatin Immunoprecipitation or ChIP-seq). These assays help identify, for example, regulators (e.g., cis regulators and/or trans regulators) that contribute to dynamic cellular phenotypes. SMT compositions could help maintain the contiguity of longer regions of accessible chromatin.

Thus, the present disclosure relates generally to the analysis of nucleic acids with split mirrored transposon compositions. In some cases, provided herein are methods that utilize a transposase enzyme to engage and fragment, for example, the accessible (e.g., open chromatin) genomic DNA and enable the simultaneous capture of DNA and RNA from a biological sample, thus revealing epigenomic insights regarding the structural features contributing to cellular regulation.

Disclosed here are methods of analyzing nucleic acids present in a biological sample. Such methods can include permeabilizing the biological sample under conditions sufficient to make the nucleic acid in the biological sample accessible to transposon insertion; providing the composition as disclosed herein and a transposase enzyme to the biological sample under conditions wherein the composition is inserted into the nucleic acid; allowing the transposase enzyme to excise the inserted transposon sequence from the nucleic acid, thereby generating a fragmented nucleic acid; and analyzing the fragmented nucleic acid as indicator of the original nucleic acid present in the biological sample.

Also provided herein are methods for determining genomic DNA accessibility including (a) contacting a transposome to a biological sample to insert transposon end sequences into accessible genomic DNA, thereby generating fragmented genomic DNA; (b) releasing one or more transposon end sequences not bound to the capture domain; (c) determining (i) all or a portion of a sequence of the fragmented genomic DNA, or a complement thereof, and using the determined sequences of (i) to determine genomic DNA accessibility in the biological sample. Exemplary methods are described in P.C.T. Publication WO 2020/047002, and U.S. Publication Nos. 20200407781 and 20210010070, each of which is incorporate in its entirety herein.

In some cases, ATAC-seq is used to generate genome-wide chromatin accessibility maps. These genome-wide accessibility maps can be integrated with additional genome-wide profiling data (e.g., RNA-seq, ChIP-seq, Methyl-Seq) to produce gene regulatory interaction maps that facilitate understanding of transcriptional regulation. For example, interrogation of genome-wide accessibility maps can reveal the underlying transcription factors and the transcription factor motifs responsible for chromatin accessibility at a given genomic location. Correlating changes in chromatin accessibility with changes in gene expression (RNA-seq), changes in transcription factor binding (e.g., ChIP-seq) and/or changes in DNA methylation levels (e.g., Methyl-seq) can identify the transcription regulation driving these changes. In disease states, there is often an imbalance in transcriptional regulation. Thus, analyzing both chromatin accessibility and, for example, gene expression using spatial analysis methods enables identification of the underlying imbalances in transcriptional regulation, and potentially the causes thereof.

In some cases, the genome-wide chromatin accessibility maps generated by spatial ATAC-seq can be used for cell type identification. For example, traditional cell type classification relies on mRNA expression levels but chromatin accessibility can be more adept at capturing cell identity. As such, the compositions disclosed herein can be used in ATAC-seq workflows as they are known in the art.

### E) Using SMTs for Spatial Analysis

Provided herein are methods using the SMT compositions described herein to examine spatial expression of a nucleic acid in a biological sample.

Non-limiting aspects of spatial analysis methodologies and compositions are described in U.S. Pat. Nos. 11,447,807, 11,352,667, 11,168,350, 11,104,936, 11,008,608, 10,995,361, 10,913,975, 10,774,374, 10,724,078, 10,640,816, 10,494,662, 10,480,022, 10,364,457, 10,317,321, 10,059,990, 10,041,949, 10,030,261, 10,002,316, 9,879,313, 9,783,841, 9,727,810, 9,593,365, 8,951,726, 8,604,182, and 7,709,198; U.S. Patent Application Publication Nos. 2020/0239946, 2020/0080136, 2020/0277663, 2019/0330617, 2020/0256867, 2020/0224244, 2019/0085383, and 2013/0171621; PCT Publication Nos. WO2018/091676, WO2020/176788, WO2017/144338, and WO2016/057552; Non-patent literature references Rodriques et al., Science 363(6434):1463-1467, 2019; Lee et al., Nat. Protoc. 10(3):442-458, 2015; Trejo et al., PLoS ONE 14(2):e0212031, 2019; Chen et al., Science 348(6233):aaa6090, 2015; Gao et al., BMC Biol. 15:50, 2017; and Gupta et al., Nature Biotechnol. 36:1197-1202, 2018; the Visium Spatial Gene Expression Reagent Kits User Guide (e.g., Rev F, dated January 2022); and/or the Visium Spatial Gene Expression Reagent Kits—Tissue Optimization User Guide (e.g., Rev E, dated February 2022), both of which are available at the 10x Genomics Support Documentation website, and can be used herein in any combination, and each of which is incorporated herein by reference in their entireties. Further non-limiting aspects of spatial analysis methodologies and compositions are described herein.

In some instances, the methods disclosed herein include methods of enhancing detection of abundance and location of a nucleic acid in a biological sample. In some instances, the methods include (a) placing the biological sample on an array comprising a plurality of capture probes, wherein a capture probe of the plurality of capture probes comprises: (i) a spatial barcode and (ii) a capture domain; (b) hybridizing the nucleic acid to the capture probe; (c) extending the capture probe using the nucleic as a template, there by generating an extended capture probe; (d) providing to the array a plurality of transposomes comprising double-stranded split mirrored transposon nucleic acid compositions, wherein a double-stranded split mirrored transposon nucleic acid composition of the plurality comprises: (i) a plurality of restriction enzyme sites comprising a first restriction enzyme site, a second restriction enzyme site, and a third restriction enzyme site, wherein the third restriction enzyme site is unique to a target genome, and wherein the third restriction enzyme site is flanked by sequences that are complementary to one another and that are capable of forming a hairpin; (ii) a first unique MI and a second MI; (iii) a first mosaic end sequence and a second mosaic end sequence; and (iv) a transposase enzyme; (e) integrating the double-stranded split mirrored transposon nucleic acid composition into the double stranded extended capture probe, and (f) determining (i) a sequence of the spatial barcode or a complement thereof, and (ii) all or a portion of a sequence of the nucleic acid, or a complement thereof, and using the determined sequences of (i) and (ii) to enhance determination of the abundance and the location of the nucleic in the biological sample compared to a method that does not utilize the plurality of split mirrored transposon nucleic acid compositions.

Also disclosed herein are methods for determining abundance and location of accessible genomic DNA in a biological sample. In some instances, the methods include (a) placing the biological sample on an array comprising a plurality of capture probes, wherein a capture probe of the plurality of capture probes comprises: (i) a spatial barcode and (ii) a capture domain; (b) providing to the biological sample a transposome comprising a double-stranded split mirrored transposon nucleic acid composition, wherein a double-stranded split mirrored transposon nucleic acid composition of the plurality comprises: (i) a plurality of restriction enzyme sites comprising a first restriction enzyme site, a second restriction enzyme site, and a third restriction enzyme site, wherein the third restriction enzyme site is unique to a target genome, and wherein the third restriction enzyme site is flanked by sequences that are complementary to one another and that are capable of forming a hairpin if in single stranded form; (ii) a first MI and a second MI; (iii) a first mosaic end sequence and a second mosaic end sequence; and (iv) a transposase enzyme; (c) integrating the double-stranded split mirrored transposon nucleic acid composition into genomic DNA, thereby generating fragmented genomic DNA; (d) binding the fragmented genomic DNA to the capture probe; and (e) determining (i) a sequence of the spatial barcode or a complement thereof, and (ii) all or a portion of a sequence of the fragmented genomic DNA, or a complement thereof, and using the determined sequences of (i) and (ii) to determine the abundance and the location of the accessible genomic DNA in the biological sample.

Capture probes on a substrate (or on a feature on the substrate) may interact with released nucleic acids through a capture domain, described elsewhere. In some cases, certain steps are performed to enhance the transfer or capture of nucleic acids to the capture probes of the array. Examples of such modifications include, but are not limited to, adjusting conditions for contacting the substrate with a biological sample (e.g., time, temperature, orientation, pH levels, pre-treating of biological samples, etc.), using a force to transport nucleic acids (e.g., electrophoretic, centrifugal, mechanical, etc.), performing amplification reactions to increase the amount of nucleic acids (e.g., PCR amplification, in situ amplification, clonal amplification), and/or using labeled probes for detecting of amplicons and barcodes.

In some cases, an array is adapted in order to facilitate nucleic acid migration. Non-limiting examples of adapting an array to facilitate nucleic acid migration include arrays with substrates containing nanopores, nanowells, and/or microfluidic channels; arrays with porous membranes; and arrays with substrates that are made of hydrogel. In some cases, the array substrate is liquid permeable. In some cases, the array is a coverslip or slide that includes nanowells or patterning, (e.g., via fabrication). In some cases where the substrate includes nanopores, nanowells, and/or microfluidic channels, these structures can facilitate exposure of the biological sample to reagents (e.g., reagents for permeabilization, biological analyte capture, and/or a nucleic acid extension reaction), thereby increasing analyte capture efficiency as compared to a substrate lacking such characteristics.

In some cases, nucleic acid capture is facilitated by treating a biological sample with permeabilization reagents. If a biological sample is not permeabilized sufficiently, the amount of nucleic acids captured on a substrate can be too low to enable adequate analysis. Conversely, if a biological sample is too permeable, nucleic acids can diffuse away from its origin in the biological sample, such that the relative spatial relationship of the nucleic acids within the biological sample is lost. Hence, a balance between permeabilizing the biological sample enough to obtain good nucleic acid migration to the substrate while still maintaining the spatial resolution of the nucleic acid distribution in the biological sample is desired. Methods of preparing biological samples to facilitate nucleic acid capture are known in the art and can be modified depending on the biological sample and how the biological sample is prepared (e.g., fresh frozen, FFPE, etc.).

After nucleic acid capture, the capture probe is extended using the captured nucleic acid as a template, thereby generating an extended capture probe. An “extended capture probe” is a capture probe with an enlarged nucleic acid sequence. For example, an “extended 3′ end” indicates that further nucleotides were added to the most 3′ nucleotide of the capture probe to extend the length of the capture probe, for example, by standard polymerization reactions utilized to extend nucleic acid molecules including templated polymerization catalyzed by a polymerase (e.g., a DNA polymerase or reverse transcriptase).

In some cases, extending the capture probe includes generating cDNA from the captured (hybridized) RNA. This process involves synthesis of a complementary strand of the hybridized nucleic acid, e.g., generating cDNA based on the captured RNA template (the RNA hybridized to the capture domain of the capture probe). Thus, in an initial step of extending the capture probe, e.g., the cDNA generation, the captured (hybridized) nucleic acid, e.g., RNA, acts as a template for the extension, e.g., reverse transcription, step. In some instances, extending the capture probe utilizes a polymerase.

In some cases, extended capture probes are amplified to yield quantities that are sufficient for analysis, e.g., via DNA sequencing. In some cases, the first strand of the extended capture probes (e.g., DNA and/or cDNA molecules) acts as a template for the amplification reaction (e.g., a polymerase chain reaction).

The biological sample comprising nucleic acids (e.g., genomic DNA and/or mRNA) is contacted to the substrate such that a capture probe can interact with the fragmented and tagged (e.g., tagmented) genomic DNA. In some cases, the biological sample comprising nucleic acids (e.g., genomic DNA, mRNA) is contacted with the substrate such that the capture probe can interact with both the tagmented genomic DNA and the mRNA present in the biological sample (e.g., a first capture probe can bind genomic DNA, a second capture probe can bind mRNA).

In some cases, the location of the capture probe on the substrate can be correlated to a location in the biological sample, thereby spatially determining the location of the nucleic acid. In some cases, the location of the capture probe on the substrate can be correlated to a location in the biological sample, thereby spatially determining the location of the genomic DNA and mRNA in the biological sample.

### Kits

Also provided herein are kits for making and using split mirrored transposons, kits for preparing a library of nucleic acids from a biological sample, kits for analyzing a nucleic acid from a biological sample, kits for analyzing a nucleic acid from a single cell biological sample, kits for enhancing detection of abundance and location of a nucleic acid in a biological sample, and kits for determining abundance and location of accessible genomic DNA in a biological sample. In some cases, the kits include (a) a double-stranded split mirrored transposon nucleic acid composition comprising: (i) a plurality of restriction enzyme sites comprising a first restriction enzyme site, a second restriction enzyme site, and a third restriction enzyme site, wherein the third restriction enzyme site is unique to a target genome, and wherein the third restriction enzyme site is flanked by sequences that are complementary to one another and that are capable of forming a hairpin; (ii) a first molecular identifier (MI) and a second MI; (iii) a first mosaic end sequence and a second mosaic end sequence; and (iv) a transposase enzyme, and (b) instructions for performing any of the methods disclosed herein.

In some cases, the kits include (a) a double-stranded split mirrored transposon nucleic acid composition comprising: (i) a plurality of restriction enzyme sites comprising a first restriction enzyme site, a second restriction enzyme site, and a third restriction enzyme site, wherein the third restriction enzyme site is unique to a target genome, and wherein the third restriction enzyme site is flanked by sequences that are complementary to one another and that are capable of forming a hairpin; (ii) a first molecular identifier (MI) and a second MI; (iii) a first mosaic end sequence and a second mosaic end sequence; and (iv) a transposase enzyme; (b) an array comprising a plurality of capture probes, wherein a capture probe of the plurality of capture probes comprises: (i) a spatial barcode and (ii) a capture domain; (c) one or more restriction enzymes; (d) one or more enzymes selected from a polymerase, a ligase, and a reverse transcriptase; and (e) instructions for performing any one of the methods disclosed herein.

### EXAMPLES

### Example 1. Method of Generating a Split Mirrored Transposon

An exemplary DNA oligonucleotide is chemically synthesized. The oligonucleotide comprises, from the 5′ end, 4 leader nucleotides, a Nt. BspQI restriction enzyme site sequence separated by 4 nucleotides from a Nb.BsrDI restriction enzyme site sequence, a degenerate molecular identifier sequence (MI), a hairpin sequence and its complement (hairpin′) separated by a Srf1 restriction enzyme site sequence (FIG. 1). The hairpin sequence and the hairpin′ sequence hybridize to one another, creating a stem-and-loop structure where the loop comprises the Srf1 restriction enzyme site sequence and the rest of the oligonucleotide, which includes the MI, and the Nt.BspQI and Nb.BsrDI restriction enzyme site sequences remain single-stranded (FIG. 2). The single-stranded oligonucleotide is extended by a polymerase to generate a double-stranded oligonucleotide, resulting in a stem-and-loop structure where the stem further includes the double stranded generation of Nt.BspQI and Nb.BsrDI restriction enzyme site sequences, and a double stranded MI section (FIG. 3). The oligonucleotide is nicked with the Nt.BspQI restriction enzyme, thereby generating a 3′ overhang. After nicking, the oligonucleotide is column purified. (FIG. 4).

A primer that includes a transposon sequence, or mosaic end, and a sequence complimentary to the 3′ overhang generated by restriction digest of the double stranded oligonucleotide with Nt.BspQI restriction enzyme is hybridized to the 3′ overhang to generate a 5′ overhang with the mosaic end. A gap remains between the 3′ of the primer and the 5′ end of the oligonucleotide after restriction digestion with the Nt.BspQI enzyme (FIG. 5). The primer is extended with a strand displacing polymerase to extend the 3′ strand of the 5′ overhang and to seal the gap, further the strand displacing polymerase displaces and processes along the oligonucleotide, unwinding the stem-and-loop structure and extending through the rest of the molecule. The strand displacing polymerase thereby generates a double-stranded oligonucleotide comprising a mosaic end adjacent to a Nb.BsrDI restriction enzyme site, a MI, a hairpin sequence, a Srf1 restriction enzyme site sequence, a hairpin′ sequence (reverse-compliment of the hairpin sequence), a second MI identical to the first, and a second Nb.BsrDI restriction enzyme site (FIG. 6).

The population of double-stranded oligonucleotides is digested with Nb.BsrDI restriction enzyme to generate a 3′ overhand on the side opposite the mosaic end, and a nick in the nucleotide backbone on the mosaic end side (FIG. 7). A duplexed mosaic end with a 5′ overhang complimentary to the 3′ overhang generated from Nb.BsrDI is ligated to the oligonucleotide. At the same time, the nick in the nucleotide backbone on the mosaic end is sealed (FIG. 8). Lastly, a transposase is complexed with the mosaic ends of the oligonucleotides to form a transposome comprising a split mirrored transposon that can function as a transposable element (FIG. 9).

### Example 2. Methods of Using a Split Mirrored Transposons

Nucleic acids of a biological sample, such as the DNA from a tissue, are extracted. Nucleic acids can be sheared, such as passing a DNA extraction solution through a needle, using sonication, etc. Nucleic acid fragments are mixed with transposomes comprising split mirror transposons (SMTs) and the nucleic acids are fragmented by digestion with Srf1 and tagged with MIs (FIG. 10A). Adaptor sequences, such as sequencing indices, sequencing primers, etc. can be appended to the ends of the fragmented and tagged nucleic acids thereby creating sequencing libraries of the nucleic acids from a biological sample (FIG. 10B).

The same methodology can be adapted for use on single cell or spatial arrays. For example, for spatial arrays tissues located on an array, wherein the array comprises capture probes that comprise spatial barcodes and capture domains, can be permeabilized to allow the transposomes disclosed herein to access the nucleic acids present in any given tissue sample, On an array, after permeabilization the transposomes are added and transposition occurs, followed by Srf1 digestion to provide a plurality of tagmented and nucleic acids, where the tagmented nucleic acid hybridizes to a capture domain on a probe attached to the array, followed by spatial transcriptomics as known in the art.

Another method which can be practiced using the SMTs as disclosed herein is exemplified in FIG. 11. Target nucleic acids can be captured directly on the array. The capture probe is extended using the captured target nucleic acid as template, and the target nucleic acid is degraded (e.g., RNAse H digestion if the target is mRNA). The extended capture probe is copied using random primer extension. The modified split-mirrored transposon complex is added and the double stranded extended capture probe is tagmented. Further, the double stranded capture probe that remains attached to the substrate can be further amplified to create copies that are no longer substrate bound. The tagmented and/or amplified nucleic acids can be removed from the sample, processed to generate sequencing ready libraries and sequenced with standard sequencing technologies. After sequencing, sequences are analyzed and bioinformatically aligned using the MIs using established methods, thereby generating sequence data that links sequencing reads, spatial barcodes, and MIs to produce long sequences where contiguity is maintained for an original DNA or RNA molecule. Further, as the capture probes on the spatial array include spatial barcodes, the sequence information is further spatially tagged as such its spatial location relative to its original location in the biological sample can be determined based from the sequencing data.

### Example 3. Method of Using a Split Mirrored Transposon in Library Preparation

A biological sample, such as a tissue sample, is permeabilized such that a nucleic acid of interest (e.g. RNA or DNA) in the biological sample is accessible to transposon insertion. Permeabilization can include chemical permeabilization, enzymatic permeabilization, or both. The split mirrored transposon and the transposase enzyme (transposome) is applied to the biological sample. The transposome inserts transposon sequences into the nucleic acids, generating at least one fragmented and tagged nucleic acid. The fragmented nucleic acids are collected, for example for generating a sequencing library. Additional methods can be used to attach various adapters or to amplify the tagmented nucleic acids that comprise molecular identifier sequences, and, optionally, used to determine all or a portion of a sequence of the tagmented nucleic acid, or a complement thereof. The determining can use high-throughput sequencing methods. The determined sequences can be used to identity the nucleic acid from which the tagmented nucleic acid originated, or the sequences can be used, for example, to quantify the abundance of a particular nucleic acid from which the tagmented nucleic acid originated.

Additionally, a biological sample can be lysed and the lysate can be used as a source of nucleic acids for transposon insertion. Further, nucleic acids for example from a lysate can be purified or partially purified away from cellular debris, wherein the purified or partially purified nucleic acids can be used as a source for transposon insertion.

### Example 4. Method of Using a Split Mirrored Transposon in Single Cell Analysis

A biological sample, such as a tissue sample, separated into single cells, which can be isolated into single cells or maintained as a plurality of single cells. A single cell is permeabilized such that a nucleic acid of interest (e.g. RNA or DNA) in the single cell is available for transposon insertion. Permeabilization can include chemical permeabilization, enzymatic permeabilization, or both. The split mirrored transposon and the transposase enzyme (transposome) is applied to the single cell, and the transposon is inserted into the nucleic acids. The transposome generates at least one tagmented nucleic acid. The tagmented nucleic acids are collected, and can be used to generate a sequencing library. Additional methods can be used to attach various adapters or to amplify the tagmented nucleic acids that comprise molecular identifier sequences, and, optionally, used to determine all or a portion of a sequence of the tagmented nucleic acid, or a complement thereof. The determining can use high-throughput sequencing methods. The determined sequences can be used to identity the nucleic acid from which the tagmented nucleic acid originated, or can be used to quantify the abundance of the nucleic acids from which the tagmented nucleic acid originated. Additionally, if a barcode is included as an adaptor or if the tagmented nucleic acids are further captured by beads that comprise a barcode that is specific to that particular cell, the cell that originated the tagmented nucleic acid can also be identified.

What is claimed is:

1. A method of generating a plurality of tagmented nucleic acid molecules, the method comprising:
(a) hybridizing a nucleic acid analyte of a biological sample to a capture probe of a plurality of capture probes affixed to an array, wherein the capture probe comprises: (i) a spatial barcode and (ii) a capture domain;
(b) extending the capture probe using the nucleic acid analyte as a template, thereby generating an extended capture probe on the array;
(c) contacting the extended capture probe or a derivative thereof on the array with a split mirrored transposon (SMT) complex, wherein the SMT complex comprises:
(i) a restriction enzyme site flanked by a first hairpin sequence on a 3′ end and a second hairpin sequence on a 5′ end, wherein the first hairpin sequence and the second hairpin sequence comprise complementary sequences and are configured to hybridize together to form a hairpin structure;
(ii) a first unique molecular identifier (UMI) sequence and a second UMI sequence, wherein the first UMI sequence and the second UMI sequence comprise complementary sequences and are configured to hybridize together;
(iii) a first mosaic end (ME) sequence and a second ME sequence, wherein the first ME sequence and the second ME sequence comprise complementary sequences and are configured to hybridize together; and
(iv) a transposase enzyme, wherein the transposase enzyme is bound to the first ME sequence and/or the second ME sequence; and
(d) digesting the restriction enzyme site using a restriction enzyme that recognizes the restriction enzyme site, thereby generating the plurality of tagmented nucleic acid molecules.

2. The method of claim 1, wherein the SMT complex comprises a double-stranded oligonucleotide, wherein the double-stranded oligonucleotide comprises in sequential order:
(a) the first ME sequence,
(b) the first UMI sequence,
(c) the first hairpin sequence,
(d) the restriction enzyme site,
(e) the second hairpin sequence,
(f) the second UMI sequence, and
(g) the second ME sequence.

3. The method of claim 1, further comprising after extending the capture probe in (b), releasing the nucleic acid analyte from the array.

4. The method of claim 3, further comprising hybridizing a primer to the extended capture probe on the array, extending the primer using the extended capture probe as a template, thereby generating a double-stranded extended capture probe.

5. The method of claim 4, further comprising tagmenting the double-stranded extended capture probe using the SMT complex, thereby generating the plurality of tagmented nucleic acid molecules.

6. The method of claim 1, wherein a tagmented nucleic acid molecule of the plurality of tagmented nucleic acid molecules comprises:
(h) the first ME sequence,
(i) the first UMI sequence,
(j) the first hairpin sequence,
(k) the second hairpin sequence,
(l) the second UMI sequence, and
(m) the second ME sequence.

7. The method of claim 6, wherein the first ME sequence and the second ME sequence each comprises an adaptor sequence or the method further comprises appending one or more adaptor sequences to the tagmented nucleic acid molecule.

8. The method of claim 1, further comprising amplifying the plurality of tagmented nucleic acid molecules.

9. The method of claim 1, further comprising determining a sequence of a tagmented nucleic acid molecule of the plurality of tagmented nucleic acid molecules, wherein the sequence of the tagmented nucleic acid molecule identifies contiguity with another tagmented nucleic acid molecule of the plurality of tagmented nucleic acid molecules.

10. The method of claim 1, wherein the capture probe further comprises a cleavage domain, a primer binding site, a third UMI sequence, or a combination thereof.

11. The method of claim 1, further comprising permeabilizing the biological sample under conditions sufficient to make the extended capture probe or the derivative thereof accessible to transposon insertion by the SMT complex.

12. The method of claim 1, wherein the transposase enzyme is a Tn5 transposase enzyme, a Mu transposase enzyme, a Tn7 transposase enzyme, a Vibhar transposase enzyme, a Mariner transposase enzyme, or a functional equivalent thereof.

13. The method of claim 1, wherein the transposase enzyme is a Tn5 transposase enzyme and comprises a sequence that has at least about 70%, 75%, 80%, 85%, 90%, or 95% sequence identity to SEQ ID NO: 1.

14. The method of claim 1, wherein the restriction enzyme is Srf1.

15. The method of claim 1, wherein the nucleic acid analyte is RNA.

16. The method of claim 1, wherein the nucleic acid analyte is DNA.

17. The method of claim 1, further comprising, before (a), contacting the biological sample with a first substrate.

18. The method of claim 17, wherein the array is on the first substrate.

19. The method of claim 17, wherein the array is on a second substrate and the method further comprises aligning the first substrate with the second substrate such that at least a portion of the biological sample is aligned with at least a portion of the array.

20. The method of claim 1, further comprising determining (i) a sequence of the spatial barcode or a complement thereof, and (ii) all or a portion of a sequence of the nucleic acid analyte or a complement thereof, wherein the sequences of (i) and (ii) identifies a location and/or an abundance of the nucleic acid analyte in the biological sample.
